(19) United States
(12) Patent Application Publication
(10) Pub. No.: US 2024/0366923 A1
(43) Pub. Date: Nov. 7, 2024

(54) FORMULATIONS OF CONTROLLED RELEASE PENETRATING MEMBERS FOR DRUG DELIVERY IN THE SMALL INTESTINE WALL

(71) Applicant: ALMA THERAPEUTICS LTD, Kfar Saba (IL)

(72) Inventors: Oz Cabiri, Hod Hasharon (IL); Anat RAZ, Herut (IL)

(21) Appl. No.: 18/688,777

(22) PCT Filed: Sep. 3, 2022
(86) PCT No.: PCT/IB2022/058299

§ 371 Date: Mar. 3, 2024

Related U.S. Application Data
(60) Provisional application No. 63/240,378, filed on Sep. 3, 2021.
(60) Provisional application No. 63/294,049, filed on Dec. 27, 2021.

(51) Int. Cl. A61M 37/00; A61K 9/00

(52) U.S. Cl. CPC A61M 37/0015; A61K 9/0065; A61M 2037/0023; A61M 2037/0061

(57) ABSTRACT

Embodiments of the invention provide solid con-trolled-release penetrating member for exposure to the intestinal lumen environment and delivery of an active agent across or within the small intestine lumen wall and having a tissue penetrating tip, especially ad-vantageous for active agents typically administered by injection. Optionally, the penetrating members include a tip coat or water insoluble extension of the controlled release formulation and/or an intestine environment protective component. Methods of using the penetrating members and arrays thereof are also provided.

### TECHNICAL FIELD

The present disclosure relates to formulations for exposure to an intestine lumen environment and subsequent delivery of an active agent across or within a wall of the intestine.

### BACKGROUND ART

Medical treatment often involves delivery of an active or therapeutic agent to patients using various means including transdermal, oral, or injectable methods. Each method has associated advantages as well as disadvantages which must be overcome in order to deliver effective therapy. For example, injections allow direct systemic delivery without involving the gastrointestinal (GI) tract and first pass metabolism. However, injections often involve pain, risk of infection, inconvenient devices for administration, forms of drug with limited stability, transport or storage capabilities, and which frequently involve overcoming the problem of patient compliance.

With conventional oral formulations, some active agents, including antibodies, proteins, and peptides, are not able to be exposed and delivered to the GI track due to the sensitivity of the molecules to mucus and gastric pH, degradation by enzymes in the GI tract, as well as the low permeation characteristics of the intestine wall for the size and polarity of this group of molecules. Thus, there is a need for new and improved methods and devices for oral delivery of proteins, antibodies, and peptides to a patient.

Orally administered, intestinal transmucosal drug delivery is an area of interest in which an active agent is delivered directly into the wall of the intestine, typically involving a solid or rigid penetrating member configured to delivery active agent without exposure to GI track. This method is of interest due to the potential of delivering systemically acting drugs with a high relative bioavailability due to increased blood flow in the area. In addition, there may be benefits to locally deliver therapeutic agents to a site in the intestine for diseases involving the intestine. One method of intestinal transmucosal drug delivery involves an orally administered transintestinal patch which does not involve pain, can deliver an active agent to the blood while also enabling smooth, consistent, and safe delivery of an active agent into the intestinal wall.

However, most of the gastrointestinal tract is covered with a defending layer of mucus which is a continuously renewed viscous fluid containing various categories of agents, such as antiseptic lysozymes, proteins, and glycoprotein mucins. The tightly lined epithelial cells with intercellular spaces sealed by tight junctions also form a barrier. These defending layers provide additional challenge to transmucosal drug delivery. First, the viscous liquid traps and slows down the intrusion of foreign matter, thus allowing time for enzymatic and other defense mechanisms to degrade and/or kill the foreign body. Secondly, the viscous liquid fluid continuously cleans and washes the surface of the mucosal tissue as it is expelled from the tissue. As such, a significant amount of drug may be wasted using conventional techniques.

Although intestinal transmucosal drug delivery has advanced over the years, there are numerous unique challenges associated with intestinal patch deployment and release of an active agent, especially macromolecules including biologics, in the intestinal wall for a period sufficient to allow for the transfer without early release of an active agent in the lumen of the intestine where it can be degraded. Attempts to provide oral delivery of biologics by incorporating protease inhibitors, mucoadhesive polymers, cross-linked enzyme crystals, liposomal encapsulation and

permeation enhancers have resulted in limited performance. In addition, intestinal transmucosal delivery devices suffer from early release of drug into the lumen and deficiencies in the penetrating member function.

An additional complexity is finding the balance between the conflicting needs of the solid penetrating member which includes the active agent. The formulation is required to protect the active agents from naturally present chemical and biologic barriers, while also promoting fast and efficient release when the delivery device is deployed at the intestine wall. It is also required to have a high degree of strength or durability to resist the conditions of the intestine lumen and preserve details of shape for effective penetration of the intestine wall. For example, many polymers used in the area of microneedles result in swelling which are not relevant for shape sensitive penetrating members. On the other hand, it must also have the ability to release a drug within enough time and prior to the disengagement from the intestine wall but preferably not too early, as this increases the risk of early and wasted drug release into the lumen. In addition, the formulative conditions for the intestine lumen environment can negatively affect release in the interstitial space of the intestine tissue. Thus, numerous challenging properties in design of these penetrating members exist. Accordingly, although transmucosal drug delivery offers certain advantages over conventional oral delivery, it would be beneficial to promote innovations in the formulation of intestine tissue penetrating members.

### SUMMARY OF INVENTION

Embodiments of the present disclosure include members, devices, systems, and methods for delivery of an active agent across or within an internally located soft tissue, for example an intestine wall. The invention further provides members, devices, systems, and methods for delivery of an active agent (e.g., especially active agents which chemically degrade, is poorly absorbed, and/or is not well tolerated in the lumen of the gastrointestinal tract) across or within the intestine wall after exposure to the intestine lumen environment for example for a period of time of at least 10 minutes. In various embodiments, the present invention provides soft tissue or intestine tissue penetrating members (such as biodegradable penetrating members) specialized for exposure to the intestine lumen environment and subsequent delivery of an active agent across or within a soft tissue and especially the small intestine lumen wall. In this case, delivery of an active agent across or within a soft tissue requires that the penetrating member is resistant to degradation by the contents of the intestinal lumen as well as intestinal mucous on the lumen wall. Typically, resistance can be quantified by defining more than 3 minutes exposure to intestinal mucous, or models thereof, without any substantial change in the morphology of the tip (e.g., diameter of tip) or alternatively without active agent release. Typically, resistance can alternatively be quantified by defining more than 8 minutes exposure to intestinal fluids (naturally occurring in the intestine), or models thereof, without any substantial change in the morphology of the tip (e.g., diameter of tip) or alternatively without active agent release. Thus, the present invention can be effective in reducing the undesired early release of the active agent into the intestine lumen (prior to penetration of the penetrating member across or within the lumen wall) and during exposure to the intestinal lumen environment (including the mucosal layer). This is especially important for active agents which may be sensitive to mechanical, biologic, or chemical degradation under typical intestine conditions. In various embodiments, the penetrating member is specifically designed and formulated such that it is resistant to degradation in the intestinal lumen environment and begins degradation and release of active agent once safely positioned in the interstitial space, a specific layer of the intestinal soft tissue. This degradation and release of active agent also needs to occur in a relatively rapid manner to ensure delivery of active agent to the blood prior to disengagement of the penetrating member but without the associated risk of early release prior to deployment at the wall.

In one aspect, there is provided, a solid controlled-release penetrating member for delivery of an active agent across or within a lumen wall of the small intestine, said member sized to be contained within an oral swallowable container and comprising an active agent, a tissue penetrating tip at a distal end thereof, and a base at a proximal end thereof, said penetrating member and/or tip shaped and formulated to promote penetration of the intestine tissue after exposure to an intestine lumen environment and upon application of a force to the base. In some embodiments, the penetrating member and/or tip shaped and formulated to promote penetration of the intestine tissue after exposure to an intestine lumen environment and upon application of a force to the base, wherein the solid tissue penetrating member is configured for exposure to the intestinal lumen environment for an exposed period of time while substantially maintaining size (and/or shape) and/or preventing release of active agent; and a subsequent controlled release of the active agent during a release period.

In one aspect, there is provided a solid controlled-release penetrating member comprising an active agent and a penetrating tip at a distal end thereof, said tip shaped and formulated to promote penetration of the intestine tissue during exposure to an intestine lumen environment (e.g., intestine fluid and/or mucous) wherein when the penetrating member is subject to simulating intestine conditions such as in vitro dissolution testing employing a USP II apparatus with dissolution medium at 37° C., 10-30 mM or preferably 30 mM buffer, pH 6.5 with fasted state simulating intestinal fluid (FaSSIF): A. at 10 minutes, i. the penetrating member or more than 80% of the penetrating member array loses less than 20% or 10% of its length as measured from base to tip, ii. the penetrating member or array of penetrating members release less than 10% of its active agent or iii. the penetrating member or more than 80% of the array of penetrating members maintains a diameter of tip of less than 30 μm or 25 μm; and B. at 120 minutes, the penetrating member releases at least 80% of its active agent.

In one aspect, there is provided an oral device for delivery of an active agent across or within the small intestine lumen wall comprising:
i. an intestine release container;
ii. a solid controlled-release penetrating member comprising an active agent and a penetrating tip at a distal end thereof, said tip shaped and formulated to promote penetration of the intestine tissue during exposure to an intestine lumen environment (e.g., intestine fluid and/or mucous) and said penetrating member being operably coupled to an intestine wall deployment assembly; and
iii. the intestine wall deployment assembly disposed in the intestine release container; said assembly configured to:
   a. position said penetrating member adjacent to the intestinal lumen wall; and
   b. apply a force to said penetrating member so as to penetrate the wall;
      wherein when the penetrating member is subject to simulating intestine conditions such as in vitro dissolution testing employing a USP II apparatus with dissolution medium at 37° C., 10-30 mM or preferably 30 mM buffer, pH 6.5 with fasted state simulating intestinal fluid (FaSSIF), the active agent is released from the penetrating member at a rate such that:
a. the penetrating member or at least about 80% of an array of penetrating members loses less than 10% of its length as measured from base to tip, releases less than 10% of its active agent; and
b. the penetrating member or array of penetrating members releases at least 80% of its active agent in 120 minutes.

In one aspect, there is provided an oral device for delivery of an active agent across or within the small intestine lumen wall comprising: i. an intestine release container; ii. a solid controlled-release penetrating member comprising an active agent and a penetrating tip at a distal end thereof, said tip shaped and formulated to promote penetration of the intestine tissue during exposure to an intestine lumen environment (e.g., intestine fluid and/or mucous) and said penetrating member being operably coupled to an intestine wall deployment assembly; and iii. the intestine wall deployment assembly disposed in the intestine release container; said assembly configured to: position said penetrating member adjacent to the intestinal lumen wall; and apply a force to said penetrating member so as to penetrate the wall; wherein when the penetrating member is subject to simulating intestine conditions such as in vitro dissolution testing employing a USP II apparatus with dissolution medium at 37° C., 10-30 mM or preferably 30 mM buffer, pH 6.5 with fasted state simulating intestinal fluid (FaSSIF): A. at 10 minutes, i. the penetrating member or more than 80% of the penetrating member array loses less than 20% or 10% of its length as measured from base to tip, ii. the penetrating member or array of penetrating members release less than 10% of its active agent or iii. the penetrating member or more than 80% of the array of penetrating members maintains a diameter of tip of less than 30 μm or 25 μm; and B. at 120 minutes, the penetrating member releases at least 80% of its active agent.

In one aspect, there is provided an oral device for delivery of an active agent across or within the small intestine lumen wall comprising: i. an intestine release container; ii. a solid controlled-release penetrating member comprising an active agent and a penetrating tip at a distal end thereof, said tip shaped and formulated to promote penetration of the intestine tissue during exposure to an intestine lumen environment (e.g., intestine fluid and/or mucous) and said penetrating member being operably coupled to an intestine wall deployment assembly; and iii. the intestine wall deployment assembly disposed in the intestine release container; said assembly configured to: position said penetrating member adjacent to the intestinal lumen wall; and apply a force to said penetrating member so as to penetrate the wall; wherein when the penetrating member is subject to simulating intestine conditions such as in vitro dissolution testing employing a USP II apparatus with dissolution medium at 37° C., 10-30 mM or preferably 30 mM buffer, pH 6.5 with fasted state simulating intestinal fluid (FaSSIF): A. at 10 minutes, the penetrating member or more than 80% of the penetrating member array loses less than 20% or 10% of its length as measured from base to tip; and B. at 120 minutes, the penetrating member releases at least 80% of its active agent.

In one aspect, there is provided an oral device for delivery of an active agent across or within the small intestine lumen wall comprising: i. an intestine release container; ii. a solid controlled-release penetrating member comprising an active agent and a penetrating tip at a distal end thereof, said tip shaped and formulated to promote penetration of the intestine tissue during exposure to an intestine lumen environment (e.g., intestine fluid and/or mucous) and said penetrating member being operably coupled to an intestine wall deployment assembly; and iii. the intestine wall deployment assembly disposed in the intestine release container; said assembly configured to: position said penetrating member adjacent to the intestinal lumen wall; and apply a force to said penetrating member so as to penetrate the wall; wherein when the penetrating member is subject to simulating intestine conditions such as in vitro dissolution testing employing a USP II apparatus with dissolution medium at 37° C., 10-30 mM or preferably 30 mM buffer, pH 6.5 with fasted state simulating intestinal fluid (FaSSIF): A. the penetrating member or array of penetrating members release less than 10% of its active agent; and B. at 120 minutes, the penetrating member releases at least 80% of its active agent.

In one aspect, there is provided an oral device for delivery of an active agent across or within the small intestine lumen wall comprising: i. an intestine release container; ii. a solid controlled-release penetrating member comprising an active agent and a penetrating tip at a distal end thereof, said tip shaped and formulated to promote penetration of the intestine tissue during exposure to an intestine lumen environment (e.g., intestine fluid and/or mucous) and said penetrating member being operably coupled to an intestine wall deployment assembly; and iii. the intestine wall deployment assembly disposed in the intestine release container; said assembly configured to: position said penetrating member adjacent to the intestinal lumen wall; and apply a force to said penetrating member so as to penetrate the wall; wherein when the penetrating member is subject to simulating intestine conditions such as in vitro dissolution testing employing a USP II apparatus with dissolution medium at 37° C., 10-30 mM or preferably 30 mM buffer, pH 6.5 with fasted state simulating intestinal fluid (FaSSIF): A. at 10 minutes, the penetrating member or more than 80% of the array of penetrating members maintains a diameter of tip of less than 30 μm; and B. at 120 minutes, the penetrating member releases at least 80% of its active agent.

In one aspect, there is provided an oral device for delivery of an active agent across or within the small intestine lumen wall comprising: i. an intestine release container; ii. a solid controlled-release penetrating member comprising an active agent and a penetrating tip at a distal end thereof, said tip shaped and formulated to promote penetration of the intestine tissue during exposure to an intestine lumen environment (e.g., intestine fluid and/or mucous) and said penetrating member being operably coupled to an intestine wall deployment assembly; and iii. the intestine wall deployment assembly disposed in the intestine release container; said assembly configured to: position said penetrating member adjacent to the intestinal lumen wall; and apply a force to said penetrating member so as to penetrate the wall; wherein when the penetrating member is subject to simulating intestine conditions such as in vitro dissolution testing employing a USP II apparatus with dissolution medium at 37° C., 10-30 mM or preferably 30 mM buffer, pH 6.5 with fasted state simulating intestinal fluid (FaSSIF): A. at 10 minutes, i. the penetrating member or more than 80% of the penetrating member array loses less than 20% or 10% of its length as measured from base to tip, ii. the penetrating member or array of penetrating members release less than 10% of its active agent or iii. the penetrating member or more than 80% of the array of penetrating members maintains a diameter of tip of less than 25 μm; and B. at 120 minutes, the penetrating member releases at least 80% of its active agent.

In general, the penetrating member continues to maintain strength, size in any dimension (e.g., length) and/or shape and/or prevent active agent release during the exposed period, i.e., while being exposed to the intestine lumen environment. Said another way, during an exposed period, the penetrating member resists chemical, biologic or mechanical degradation or diffusion, or the loss of structural integrity of the formulation as a whole.

Thus, in various embodiments of any of the aspects presented, the solid controlled-release penetrating member, when subject to in vitro dissolution testing using simulating intestine conditions for an exposed period of more than 1 minute, more than 3 minute, more than 5 minutes, more than 8 minutes, more than 10 minutes, more than 15 minutes, more than 20 minutes or between 1 and 30 minutes, between 1 and 20 minutes, between 1 and 10 minutes, 5 and 30 minutes, between 5 and 20 minutes, between 5 and 10 minutes, 10 and 30 minutes, between 10 and 20 minutes, 15 and 30 minutes, between 15 and 20 minutes, or between 10 and 15 minutes, there is substantially no release of active agent or substantially no change in the size of the penetrating member.

In various embodiments of any of the aspects presented, the present disclosure provides solid controlled-release penetrating member (“penetrating member”) and systems thereof wherein the active pharmaceutical ingredient (“API”) or active agent release is controlled until exposure to the target layer in the tissue, the interstitial space or lamina propria layer, for example, in the small intestine lumen wall. In some embodiments, the active agent is released at a rate, preferably within 15 minutes to three hours from delivery and exposure to the intestinal lumen. Said another way, the solid controlled-release penetrating member is resistant to active agent release as measured for example using in vitro simulated or ex vivo simulated models for a protected period of time and provides a controlled or controlled release of an active agent during a controlled release period of time. The controlled or controlled release of an active agent is at a decreased rate compared to a standard immediate release profile of an oral formulation or transdermal immediate release formulation. The controlled release of an active agent is at an increased rate compared to a standard transdermal microneedle depot as measured in blood or an in vitro simulated or ex vivo simulated model.

In various embodiments of any of the aspects presented, the tissue solid controlled-release penetrating member, when subject to in vitro dissolution testing employing a USP II apparatus with dissolution medium at 37° C., 30 mM buffer, pH 6.5 to 7.5 with fasted state simulating intestinal fluid (FaSSIF) for 10 minutes, substantially no active agent is released.

In various embodiments of any of the aspects presented, the tissue solid controlled-release penetrating member or tip thereof, when subject to in vitro dissolution testing employing a USP ∥ apparatus with dissolution medium at 37° C., 30 mM buffer, pH 6.5 to 7.5 with fasted state simulating intestinal fluid (FaSSIF) for 10 minutes, substantially no change in any one dimension or substantially no change in tip diameter or less than 20% or 10% or 5% or 3% or 1% change in size compared to the starting size of any dimension. In this context, when an array of penetrating members is employed, more than 80% have substantially no change in any one dimension or substantially no change in tip diameter.

In various embodiments of any of the aspects presented, the solid controlled-release penetrating member is an array or a plurality of penetrating member (e.g., microneedles). In some embodiments, the solid controlled-release penetrating member array has an active agent in an amount which makes up a portion of a therapeutically effective dose of at least one therapeutic agent or active agent. In some embodiments, the total amount of therapeutic agent within the array to produce a desired therapeutic effect is less than an amount to produce a corresponding effect if the agent was orally delivered in a capsule without the oral device. In some examples, the total active agent in the array of members is in an amount of more than 2 mg, more than 4 mg, more than 6 mg, more than 8 mg or in an amount of between 2-10 mg. In some embodiments, the active agent is in solid, semi-solid or liquid form within the solid penetrating member.

In various embodiments of any of the aspects presented, the tissue solid controlled-release penetrating member of any one of the aspects, comprises active agent loaded microparticles contained or shaped as a microneedle. In some embodiments, the penetrating member comprises a first molding material. In some embodiments, the penetrating member comprises a mixture of a) a polymer first molding material and b) a second molding material comprising particles (e.g., rigid particles or microparticles) dispersed therein, wherein the first molding material is micro molded into the plurality of microneedles. In some embodiments, the penetrating member comprises an enteric coating or layer and a molding material comprising rigid particles dispersed therein, wherein the enteric coating is placed in the mold and a molding material with rigid particles are micro molded into the plurality of microneedles.

In some embodiments of any of the aspects presented, a solid controlled-release penetrating member is operatively connected to an intestine wall deployment assembly. A number of intestine wall deployment assemblies are known in the art including for example U.S. Publication No. 2020/0276426 filed Feb. 28, 2020, and U.S. Pat. No. 10,980,750, filed on Jun. 8, 2020, both applications herein incorporated by reference. In some examples, the penetrating members are bound to a rigid or pliable substrate. In some embodiments, multiple penetrating members are each connected to a common pliable substrate at respective bases of the multiple penetrating members. In other embodiments, the penetrating member is configured to be bound to a pliable substrate which is folded to be contained in a container and to be delivered from the container to penetrate and be advanced into the lumen wall by the application of force on the penetrating member or substrate thereof. In some examples, the total force per area applied to the penetrating members or substrate is about 2 to 5 psi (pound force per square inch).

In some applications, the shape of the expandable member in its expanded form takes up a partial volume of the intestine at that location and applies a force on only part of the circumference of the inner wall of the intestine thereby serving as a temporary anchor that holds the drug delivery device in position for penetration of the penetrating members. The expandable member does not apply force to facing intestinal walls along axial extents thereof.

In various embodiments, the solid controlled-release penetrating member further comprises an outer coating of intestine environment protective component. In various embodiments, the intestine environment protective component surrounds or coats the controlled release coating. In various embodiments, the tip is at least partially coated, or the tip is formulated as a separate but adjacent and distal extension of the active agent formulation. In various embodiments, the solid controlled-release penetrating member comprises an intestine environment protective component and a tip coating. In some embodiments, the active agent is specifically released in the general area of the interstitial space of the intestine due to the choice of intestine environment protective component which may also target the pH specific to the interstitial space.

In various embodiments, the controlled release formulation includes an active agent and controlled release component. In some embodiments, the solid controlled-release penetrating member comprising the active agent is i. a mixture of active agent and controlled release component; or ii. an immediate release active agent inner core and controlled release coating.

The control release coating may include pH sensitive (e.g., cationic acrylic/methacrylic copolymer or acrylic/methacrylic copolymer having a pH threshold at about 6.5) or sustained release polymers or excipients.

Sustained release polymers include for example lipophilic coatings. Lipophilic coatings may include an excipient selected from the list consisting of: polyglycolide (PGA), polylactide (PLA), poly-epsilon-caprolactone, poly dioxanone (a polyether-ester), poly lactide-co-glycolide, polyamide esters, polyalkalene esters, polyvinyl esters, polyvinyl alcohol, and polyanhydrides.

Typically, in various embodiments, coatings mentioned throughout, are located in a layer externally located relative to the active agent inner (e.g., inner core), and form the tip coating, controlled release coating or intestine environment protective component. Coatings may have a width of between 2 and 25 μm and preferably less than 10 μm. In addition, one may measure a combined thickness of multiple layers of coatings, for example, the outer control release coating and intestine environment protective component and the combined thickness may be less than 50 μm.

In some embodiments, the immediate release active agent inner core can be a hollow shell with dry active agent, or an active agent admixed with a biodegradable excipient or polymer. For example, immediate release active agent inner core may include any one or more biodegradable polymers having monomer units designed for drug delivery. Relevant examples of biodegradable polymers include but are not limited to: natural polymers; polyesters including poly(lactic acid) (“PLA”), poly(lactic-co-glycolic acid) (“PLGA”), poly(caprolactone) (“PCL”), poly(glycolic acid) (“PGA”); chitosan; poly(ortho esters) such as (POE I, POE II, POE Ill or POE IV); hyaluronic acid; poly(anhydrides); poly(amides); poly(ester amides); poly(phosphoesters); poly(alkyl cyanoacrylates) (“PACA”). Additional excipients for immediate release of an active agent are also possible and can include for example dextrin, polysaccharides, arginine, carboxymethylcellulose, water miscible polymers including polyethylene glycol (“PEG”), polyvinyl alcohol (“PVA”) or polyvinylpyrrolidone (“PVP”) (optionally in combination with polymers containing a cationic acrylic/methacrylic copolymers, said polymers being based on methacrylic acid esters and a small portion of trimethyl aminoethyl methacrylate chlorideⁱ [such as, Eudragit RS 100®] or based on meth acryl acid esters and a small portion of amino methacrylate copolymers [such as Eudragit E 100®]. Other options are also possible and can be chosen by those skilled in the art.

The active agent inner core can further comprise a stabilizer, buffer, polymer, antioxidant, diluent, lubricant, binder, or plasticizer and can be chosen by those skilled in the art. Surfactant can also be added and can be especially important to contribute to structural stability and reduce exposure of hydrophobic regions so as to decrease or limit interface-induced aggregation and protein-protein interaction and resulting mechanical stress. Examples include poloxamers, polysorbate 20 and especially polysorbate 80. Relevant ratios to active agent may depend on the active agent and can be calculated.

In some embodiments, the mixture comprises a controlled release component such as a non-disintegrating carrier matrix selected from the list consisting of acacia, sodium alginate, gelatin, carboxymethyl cellulose sodium, methylcellulose, ethylcellulose, cellulose acetate or polyacrylates (e.g., ammonio methacrylate copolymers i.e., Eudragit RS/RL), polyethylene glycol (PEG), polyvinyl polymers, methylcellulose (e.g., HPMC, HPC) and ethylcellulose.

In another embodiment, the mixture comprises a controlled release component selected from the list consisting of polyvinyl polymers, methylcellulose and ethylcellulose. The ethyl cellulose may be a low-viscosity ethyl cellulose. When a low-viscosity ethyl cellulose is used, the mixture further comprises a pore-forming agent selected from the group consisting of hydroxypropyl cellulose and hydroxypropyl methylcellulose. The matrix may additionally include further excipients such as binders, fillers, process enhancers such as lubricants and glidants. In some embodiments, the excipients do not include a disintegrant.

In some embodiments of any of the aspects presented, the one or more solid controlled-release penetrating member are at least partially covered with polymer and specifically a slow-release polymer which delays exposure of the penetrating member. Examples include a pH-dependent enteric coating having sensitivity at pH greater than 6.0, PLGA, PLGA/PEG, PLGA/PAA, an aqueous pore forming film (water soluble ethyl cellulose and water soluble hydroxypropyl cellulose), polymer or semi-permeable film (e.g., combination of PVP K30 and hydrophilic polyethylene glycol such as PEG 6000), silk fibroin, a lipophilic (PCL, PDO, HCO) or hydrophobic polymer. This coating may additionally include pore formers, plasticizers, and processing enhancing agents such as lubricants.

Suitable the pore formers are known in the art and include HPC, HPMC, polyethylene glycol, poloxamer, povidone or a saccharide. Preferably, it is HPC, HPMC, polyethylene glycol, or povidone. Suitable amounts of pore formers in the coating polymer to pore former of 5:1 to 2:1 or 5:1 to 1:1 and preferably ratios of 100:35 to 100:45.

In various embodiments, the solid controlled-release penetrating member includes an outer control release coating comprises any suitable control release coating forming polymers which enable diffusion-based controlled release known in the art for instance: polyacrylates such as ammonio methacrylate copolymers (Eudragit RS/RL), polyvinylacetate, hydroxypropyl methylcellulose (optionally with ethylcellulose), carboxymethylcellulose, hydroxypropyl cellulose, polyvinyl pyrrolidone (optionally with carboxymethylcellulose), alginates (e.g., sodium alginate or alginate/chitosan), methylcellulose, lipophillic poly-ε-caprolactone and ethyl cellulose. In a preferred embodiment, the outer controlled release coating is ethylcellulose, cellulose acetate, or polyacrylates such as ammonio methacrylate copolymers (Eudragit® RS or RL), polyvinylacetate or combinations thereof. The coating further includes a plasticizer. The amount of plasticizer is typically between 8 and 35% or preferably 10 to 25% relative to the control release polymer.

In some embodiments, the control release coating comprises a low-viscosity ethyl cellulose, a pore-forming agent, and a plasticizer. The low-viscosity ethyl cellulose and a pore-forming agent may be present in a ratio of the pore-forming agent to low-viscosity ethyl cellulose of about 9:1 to about 1:1. The low-viscosity ethyl cellulose and a pore-forming agent may be present in a ratio of the pore-forming agent to low-viscosity ethyl cellulose of about was 9:1 to 5:5 or 7:4 to 4:5. Relevant ratios include but are not limited to 9:1, 8:2, 6:4, 5:5 and 4:5. In some cases, control release coating having a low-viscosity ethyl cellulose may further comprise a plasticizer such as triethyl citrate.

In some embodiments, the control release coating comprises a hydroxypropyl cellulose or hydroxypropyl methylcellulose having a viscosity of about 4 cP to about 14 cP.

In some embodiments, the solid controlled-release penetrating member has an outer control release coating which is a sustained release or cationic acrylic/methacrylic copolymer.

Thus, in some embodiments, the acrylic/methacrylic copolymer is cationic. In some embodiments, the acrylic/methacrylic copolymer is non-biodegradable. In some embodiments, the acrylic/methacrylic copolymer has a high or low permeability.

In some embodiments, the acrylic/methacrylic copolymer has high permeability. The (meth)acrylate copolymer may be composed of 50 to 70% by weight of methyl methacrylate, 20 to 40% by weight of ethyl acrylate and more than 7 up to 12% by weight of a salt of 2-trimethylammoniumethyl methacrylate, preferably 2-trimethylammoniumethyl methacrylate chloride (Eudragit® RL type). More specifically, a suitable copolymer comprises 65% by weight of methyl methacrylate, 30% by weight of ethyl acrylate and 10% by weight of 2-trimethylammoniumethyl methacrylate chloride. The glass transition temperature of said copolymer is about 70° C. Specific example include but are not limited to Eudragit RL 100®, Eudragit RL PO®, Eudragit RL 12.5® and Eudragit RL 30D®.

In some embodiments, the acrylic/methacrylic copolymer has low permeability. The (meth)acrylate copolymer may be composed of 50 to 70% by weight of methyl methacrylate, 20 to 40% by weight of ethyl acrylate and 7 to 2% by weight of a salt of 2-trimethylammoniumethyl methacrylate, preferably 2-trimethylammoniumethyl methacrylate chloride. The glass transition temperature of said copolymer is about 65° C. Specific examples include but are not limited to Eudragit® Type RS and specifically Eudragit RS 100®.

In some embodiments, the controlled release coating comprises an acrylic/methacrylic copolymer and further comprises a pore former. In some embodiments, the partial coating is a non-swelling polymer. In some embodiments, the partial coating has a thickness of between 2 and 50 μm. In some embodiments, the partial coating has a thickness of between 2 and 25 μm. In some embodiments, the partial coating has a thickness of less than 10 μm.

In some embodiments, the solid controlled-release penetrating member includes a multicompartment or double coating structure. The double coating structure may include a controlled release component as a first acrylic/methacrylic copolymer (for example a Eudragit RL 100®, Eudragit RL PO®, Eudragit RL 12.5®, Eudragit RL 30D®, Eudragit RS 100®) and an additional outer coating of intestine environment protective component is an enteric acrylic/methacrylic copolymer (for example, Eudragit S 100®, a combination of Eudragit S 100® and Eudragit L 100®, Eudragit S 12.5®).

In a particular embodiment of the invention, the inner active agent core can be layered with a coating such that the drug is released from the formulation specifically in the interstitial space of the intestine tissue. Thus, in some embodiments, the outer control release coating is a pH dependent film-forming polymer having a pH threshold at about pH 6.5 or above. These film-forming polymers are typically used in targeted delivery of an active agent to the colon where the pH is higher than the small intestine lumen however, in this case, it is used to provide a targeted release of the active agent to the interstitial space of the intestine tissue after penetration of the penetrating member.

In specific embodiments of the outer control release coating, the pH dependent film-forming polymer is an acrylate polymer. The acrylate polymer may be an anionic copolymer of (meth)acrylic acid and (meth)acrylic acid C₁₋₄ alkyl ester. The acrylate polymer may be a copolymer of methacrylic acid and ethyl acrylate. The acrylate polymer may be an anionic co-polymer of methacrylic acid and methacrylic acid methyl ester. The acrylate polymer may have a ratio of methacrylic acid to methacrylic acid methyl ester is about 1:1 to 1:2. Preferably, the acrylate polymer may have a ratio of methacrylic acid to methacrylic acid methyl ester is about 1:2. The acrylate polymer may be an have pH threshold is of about 7 meaning that it is insoluble below pH 6.5 and, more preferably, is insoluble below pH 7. The acrylate polymer may be an anionic poly(methacrylic acid/methyl methacrylate) copolymer with an acid to ester ratio is of 1 to 2, and/or a molecular weight of approximately 135,000 and/or and a pH threshold is of about 7 such as Eudragit S100® or Eudragit S 12.5 or mixtures thereof. Other examples include other copolymers such as methyl methacrylate and methacrylic acid such as Eudragit® FS30D. The coating can also be a mixture and include an additional methacrylic acid and ethyl acrylate copolymer such as one which has a pH threshold of about 6.0 including for example a methacrylic acid and methyl methacrylate copolymers (1:1 ratio) such as Eudragit L-100® or Eudragit L12.5®.

Other pH dependent film-forming polymer having a pH threshold at about pH 6.5 or above may also be used. Examples include cellulose polymers or a polyvinyl-based polymers.

For example, a cellulose polymer can be cellulose acetate phthalate (“CAP”), cellulose acetate trimellitate (“CAT”) or hydroxypropyl methylcellulose acetate succinate. Specifically, hydroxypropyl methylcellulose acetate succinate (“HPMC-AS”) such as type H which has a low succinoyl to acetyl group ratio or hydroxypropyl methylcellulose phthalate (HPMCP) such as HP-55 grade can also be used. Exemplary polyvinyl-based polymers may include polyvinyl acetate phthalate (“PVAP”). In addition, shellac (for example SSB® Aquagold).

In various embodiments of any of the aspects presented, the solid controlled-release penetrating member includes a tissue penetrating shape. In some embodiments, the shape is a tip having a sharp edge and shaped to promote penetration of soft tissue or more specifically, intestine tissue and resist degradation during exposure to an intestine lumen environment. In some embodiments, the shape includes a distal tip having mechanical strength to support penetration though the mucosal layers of the intestine and reach the interstitial space. In some embodiments, the mechanical strength of the tip remains substantially consistent during the intestinal lumen exposure period such that penetration though the mucosal layer of the intestine is smooth or efficient. Said another way, the tip resist degradation during exposure to an intestine lumen environment.

Thus, in various embodiments of any of the aspects presented, the tissue penetrating shape or distal tip of the solid controlled-release penetrating member is coated or covered on the outside with a protective layer or coating. Alternatively, the tip is formulated as a distinct section at the distal end of the penetrating member such that it is slow dissolving and distinct from the formulation having an active agent. In this case, the tip has a distinct formulation and is an adjacent and distal extension of the controlled release mixture or coated inner immediate release active agent. Thus, resistance to degradation or mechanical strength especially under conditions of the intestinal lumen, is preserved by the protective coating, covering, or the distinct distal end.

Application of the at least partial coating or layer may be via spray coating, 3D printing, dipping, dropping, inkjet or immersion. The fabrication of the slow dissolving tip section may be by pouring the formulation into a mold, centrifuging, and allowing to set prior to adding an additional layer or coating with active pharmaceutical. Both the additional layer and the coating can provide additional strength required for supporting the piercing of soft tissue. Relevant materials for formulation include any pharmaceutically acceptable water insoluble, hydrophobic polymers, or non-degradable polymers including but not limited to an alginic acid and salts thereof, polyvinylpyrrolidone (e.g., Polyvidone® CL, Kollidon® CL, Polyplasdone® XL-10), poly(lactic-co-glycolic acid, thermoplastic polyester such as Polylactic acid, or polyvinyl acetate copolymer.

In various embodiments, the intestine environment protective component is associated with one or more tissue solid controlled-release penetrating member s whether formulated within the penetrating member or in at least a partial layer or coating surrounding the active agent. The intestine environment protective component may be configured to protect the penetrating member from degradation (e.g., from the environment of the gastrointestinal tract) until it is advanced into tissue.

In some embodiments, the intestine environment protective component can undergo time dependent degradation during an exposed period under the pH conditions of the intestine lumen. For example, the intestine environment protective component may be configured to protect the tissue solid controlled-release penetrating member and thereby substantially reduce or eliminate a risk of premature release of a drug where it is not readily absorbed; thus, such an intestine environment protective component of the penetrating member may help retain the treatment efficacy of the dose of drug provided in the drug delivery device.

The intestine environment protective component can form at least a partial outer coating on the controlled release coating. It may include a lipophilic coating including an excipient selected from the list consisting of: polyglycolide (PGA), polylactide (PLA), poly-epsilon-caprolactone, poly dioxanone (a polyether-ester), poly lactide-co-glycolide, polyamide esters, polyalkalene esters, polyvinyl esters, polyvinyl alcohol, and polyanhydrides) or a film-forming polymeric material having a pH threshold at about 6.5 or above. In specific embodiments, it may include PLGA or PLGA/PEG or PLGA/PAA, an aqueous pore forming film (water soluble ethyl cellulose and water soluble hydroxypropyl cellulose), a polymer or semi-permeable film (e.g., combination of PVP K30 and hydrophilic polyethylene glycol such as PEG 6000), or a silk fibroin or a lipophilic (PCL, PDO, HCO) or hydrophobic polymer. In some examples, the layer or coating is at least a partial coating provided by spray coating, dipping, dropping, inkjet, immersion. In some examples, the layer or coating is a film comprising HPMC (for example HPMC E15), plasticizer (such as propylene glycol) and a disintegrating agent.

In some embodiments of the invention, the intestine environment protective component is a pH dependent film-forming polymer having a pH threshold at about pH 6.5 or above or specifically having a threshold of about 7.0. These film-forming polymers are typically used in targeted delivery of an active agent to the colon where the pH is higher than the small intestine lumen however, in this case, it is used to protect from early release in the stomach and small intestine but provide an additional and novel effect of targeting release of the active agent to the interstitial space of the intestine tissue after penetration of the penetrating member.

In specific embodiments of the intestine environment protective component, the pH dependent film-forming polymer is an acrylate polymer. The acrylate polymer may be an anionic copolymer of (meth)acrylic acid and (meth)acrylic acid C₁₋₄ alkyl ester. The acrylate polymer may be a copolymer of methacrylic acid and ethyl acrylate. The acrylate polymer may be an anionic co-polymer of methacrylic acid and methacrylic acid methyl ester. The acrylate polymer may have a ratio of methacrylic acid to methacrylic acid methyl ester is about 1:2. The acrylate polymer may be an anionic poly(methacrylic acid/methyl methacrylate) copolymer with an acid to ester ratio is of 1 to 2, and/or a molecular weight of approximately 135,000 and/or and a pH threshold is of about 7 such as Eudragit S100® or Eudragit S 12.5 or mixtures thereof. Other examples include other copolymers such as methyl methacrylate and methacrylic acid such as Eudragit® FS30D. The coating can also be a mixture and include an additional methacrylic acid and ethyl acrylate copolymer such as a methacrylic acid and methyl methacrylate copolymers (1:1 ratio) such as Eudragit® L-100®.

Other pH dependent film-forming polymer having a pH threshold at about pH 6.5 or above may also be used. Examples include cellulose polymers or a polyvinyl-based polymers.

For example, a cellulose polymer can be cellulose acetate phthalate (“CAP”), cellulose acetate trimellitate (“CAT”) or hydroxypropyl methylcellulose acetate succinate. Specifically, hydroxypropyl methylcellulose acetate succinate (“HPMC-AS”) such as type H which has a low succinoyl to acetyl group ratio or hydroxypropyl methylcellulose phthalate (HPMCP) such as HP-55 grade can also be used. Exemplary polyvinyl-based polymers may include polyvinyl acetate phthalate (“PVAP”). In addition, shellac (for example SSB® Aquagold).

In some embodiments, the intestine environment protective component can undergo ion, time, or pH dependent degradation during a release period under the pH conditions of the intestine lumen.

In various embodiments, the present disclosure provides solid controlled-release penetrating member s specifically configured to meet the requirements of an intestine wall deployment assembly. An example of a device is referenced in U.S. Pat. No. 10,675,248, filed Aug. 14, 2018, and incorporated herein by reference.

In some embodiments, the intestine wall deployment assembly is configured to position the penetrating member adjacent to the intestinal lumen wall; and apply a force to said penetrating member so as to penetrate the wall. In some embodiments, the intestine wall deployment assembly is configured to deploy the penetrating member at the wall of the intestine lumen within 15 minutes of passing the pyloric valve or within 15 minutes of exposure to the small intestinal environment after at least partial release from an intestine release container, such as an enteric capsule. The oral device for delivery of active agent across or within a lumen of the small intestine is configured to subsequently apply a force to the penetrating members which are shaped to promote penetration and eventually penetrate the intestine tissue. In addition, the penetrating members of the present invention are specifically configured to meet the requirements of an oral device for delivery of active agent across or within a lumen of the small intestine which is maintained adjacent to the intestine lumen wall for more than 15 minutes or more than 30 minutes or about 15 minutes to 240 minutes or about 15 minutes to about 120 minutes.

In some embodiments, intestine wall deployment assembly is deployed adjacent to the intestinal lumen wall within 15 minutes of exposure to the small intestine environment. In some embodiments, the intestine wall deployment assembly is configured to be deployed in the lumen of the intestine within 15 minutes of exposure to the small intestinal environment and for about 240 minutes. In some embodiments, the intestine wall deployment assembly is configured to be deployed in the lumen of the intestine within 15 minutes of exposure to the small intestinal environment and for about 180 minutes. In some embodiments, the intestine wall deployment assembly is configured to be deployed in the lumen of the intestine within 15 minutes of exposure to the small intestinal environment and for about 120 minutes.

In various embodiments, preventing release of an active agent or maintenance of the size and/or shape of the solid controlled-release penetrating member is for an exposed period wherein less than 10% of the active agent is released after exposure to the intestinal environment and prior to penetration, for example within 15 minutes of release from the intestinal release container or exposure to the small intestine lumen environment. In some embodiments, more than 90% is released during the release period while the penetration member is positioned in the wall. A typical release period may be within 10 minutes, 20 minutes, 30 minutes, 40 minutes, 50 minutes, 60 minutes, 70 minutes, 80 minutes, 90 minutes, 100 minutes, 120 minutes, 180 minutes, or 240 minutes of exposure to the intestinal environment.

In various embodiments of any of the aspects presented, exposure to the intestinal lumen environment is for an exposed period of time prior to provide for intestine luminal wall deployment of the one or more penetrating members. The exposed period of time to the intestinal lumen environment may be greater than 1 minute, greater than 5 minutes, greater than 10 minutes, greater than 15 minutes, greater than 20 minutes, greater than 25 minutes or greater than 30 minutes.

In other embodiments, the exposed period of time to the intestinal lumen environment is between 5 and 30 minutes, between 10 and 30 minutes, between 15 and 30 minutes or between 20 minutes and 30 minutes. In some embodiments, the exposed period of time is greater than 5 minutes or less than 15 minutes. Combinations of the above-referenced ranges are also possible (e.g., between 15 and 20 minutes). Other ranges are also possible.

In certain embodiments of any of the aspects or embodiments presented, the release period of time for the active agent is sufficient to ensure that most of the active agent is deposited within the intestinal wall such that active agent does not chemically degrade in the intestinal lumen environment. In some embodiments, the release period of time (i.e., the time for release of the active agent from the one or more penetrating members) may be greater than 10 minutes, greater than 20 minutes, greater than 30 minutes, greater than 45 minutes, greater than 1 hour, greater than 2 hours or greater than 3 hours or greater than 4 hours. In some embodiments, the release period of time (e.g., during which there is release to the interstitial space) is between 10 minutes and 5 hours, 20 minutes and 5 hours, 30 minutes and 5 hours, 1 hour and 5 hours, 10 minutes and 4 hours, 20 minutes and 4 hours, 30 minutes and 4 hours, 1 hour and 4 hours, 10 minutes and 3 hours, 20 minutes and 3 hours, 30 minutes and 3 hours, 1 hour and 3 hours, 10 minutes and 2 hours, 20 minutes and 2 hours, 30 minutes and 2 hours or 60 minutes and 120 minutes, 10 minutes and 90 minutes, 20 minutes and 90 minutes, 30 minutes and 90 minutes or 60 minutes and 90 minutes or 10 minutes and 60 minutes, 20 minutes and 60 minutes, 30 minutes and 60 minutes. Combinations of the above-referenced ranges are also possible (e.g., greater than 15 to 30 minutes) Other ranges are also possible.

In certain embodiments of any of the aspects or embodiments presented, the release period of time for the active agent can be measured by a change in size such that there is less than 15% change or less than 10% or less than 8% or less than 5% or less than 3% or less than 2% change in size of the penetration member in any dimension during an exposed period. In certain embodiments of any of the aspects or embodiments presented, the release period of time for the active agent can be measured by a change in weight such that less than 15% change or less than 10% or less than 8% or less than 5% or less than 3% or less than 2% change in weight per penetrating member is measured.

In any of the embodiments described herein, the intestine wall deployment assembly may apply minimal or no force to facing intestinal walls when deployed in the lumen of the intestine and under conditions without intestinal peristalsis. Said another way, the force of opposing intestinal walls does not cause the penetration of the penetrating members. In some embodiments, the intestine wall deployment assembly is configured to be deployed in the lumen of the intestine and apply no force to facing intestinal walls.

In various embodiments of the present invention, the intestine wall deployment assembly comprises i. an expandable member that is associated with one or more solid controlled-release penetrating member, said expandable member having proximal and distal surfaces that face in generally opposite directions and configurable in a compacted configuration, an unconstrained configuration, and an expanded configuration; ii. a gas generating compartment in fluid communication with the expandable member and having at least a water or humidity permeable window, housing a gas generating formulation comprising two reactants which produce gas in the presence of water (e.g., sodium bicarbonate and citric acid) and configured to expand the expandable member (e.g., pliable expandable member) from an unconstrained configuration to an expanded configuration; and iii. one or more solid controlled-release penetrating member described in various embodiments, operably coupled to said expandable member and extending from the proximal surface thereof. In some examples, the one or more penetrating members are sized and configured to penetrate a mucosal barrier of the intestinal wall to release an active agent and comprising: an active agent, an intestine environment protective component for maintaining strength and/or shape and/or resisting active agent release during exposure to the intestinal lumen environment for a period of time and an active agent release component for subsequent release of the active agent across of within the small intestine lumen wall for a period of time to release the active agent.

In another aspect of the present invention, there is provided a method of penetrating the mucosal tissue to deliver an active agent comprising: converting a pliable expandable member from an constrained configuration to an expanded configuration, said expandable member being operably connected to one or more solid controlled-release penetrating member in an intestinal protected from comprising a layer or film or pore forming polymer (e.g., a plurality of microneedles) which are exposed to the intestine environment for a period of time; enzymatic, chemical, or mechanical degradation of a layer or film or pore forming polymer to expose an exposed form of the one or more penetrating members; penetrating the mucosal barrier with one or more solid controlled-release penetrating member; and releasing active agent over a release period of time. In some embodiments, the exposed period of time is greater than 5 minutes or less than 15 minutes. In some embodiments, the release period of time is between 15 minutes and 2 hours.

In another aspect of the present invention, there is provided a method of delivering an active agent across or within the small intestine lumen wall, the method comprising the steps of: deploying a drug delivery device (e.g., patch) adjacent to a small intestine lumen wall said drug delivery device comprising one or more solid controlled-release penetrating member s (e.g., a plurality of microneedles), a pliable base and an expandable member; exposing the one or more penetrating members (e.g., a plurality of microneedles), pliable base and an expandable member to the intestinal fluid for a period of time and subsequently, penetrating the mucosal tissue with one or more solid controlled-release penetrating members (e.g., a plurality of microneedles) that extend from, or are extendable from, the expandable member at a selected time after the drug delivery device is deployed in the lumen; and releasing an active agent in a controlled manner over a period of between 15 minutes and 4 hours such that the active agent is delivered through the plurality of microneedles into the mucosal tissue.

In another aspect of the present invention, there is provided a method of delivering an active agent across or within the small intestine lumen wall, the method comprising the steps of: receiving, by a subject, an oral device for delivery of active agent across or within a lumen of the small intestine comprising: (i) an outer intestine release container and (ii) a drug-delivery device contained within the release component, which includes an active agent formulation shaped as a penetrating members and configured to be advanced across or within the small intestine lumen wall; swallowing the drug delivery device by the subject; degrading or dissolving the outer intestine release container in the presence of an intestinal lumen environmental condition; exposing the solid controlled-release penetrating member to the intestine environment for a period of time while preventing release of the active agent and/or maintaining strength and/or maintaining shape of the solid controlled-release penetrating member to advance the at least one penetrating member across or within the small intestine lumen wall tissue; and release of the active agent into the wall of the small intestine lumen over a period of time.

In another aspect of the present invention, there is provided a method of delivering an active agent across or within the small intestine lumen wall, the method comprising the steps of: receiving, by a subject, an oral device for delivery of active agent across or within a lumen of the small intestine comprising: (i) an outer intestine release container and (ii) a drug-delivery device contained within the release component, which includes an active agent formulation shaped as a penetrating member for advancing a penetrating member across or within the small intestine lumen wall, the expandable member expanded by gas generating compartment; configured to penetrate a the mucosal barrier of the small intestine lumen wall, an expandable member configurable in a compacted configuration, an unconstrained configuration and an expanded configuration; swallowing the drug delivery device by the subject; degrading or dissolving the outer intestine release container in the presence of an intestinal lumen environmental condition; exposing the solid controlled-release penetrating member to the intestine environment for a period of time while preventing release of the active agent and/or maintaining strength and/or maintaining shape of the penetrating member to allow for a period of time for the expandable member to reach an expanded configuration to advance the at least one penetrating member across or within the small intestine lumen wall tissue; and release of the active agent across or within the small intestine lumen wall over a period of time.

In another aspect of the present invention, there is provided a method for delivering an active agent across or within the small intestine lumen wall, the method comprising the steps of: receiving, by a subject, oral device for delivery of active agent across or within a lumen of the small intestine comprising: (i) an outer intestine release container and (ii) a drug-delivery device contained within the release component, which includes (a) an expandable member for advancing a penetrating member across or within the small intestine lumen wall, the expandable member expanded by gas generating compartment; and a penetrating member; and swallowing the drug delivery device by the subject; dissolving the outer intestine release container (e.g., enteric layer as capsule or coated soft gel capsule) in the small intestine; exposing a first state of the solid controlled-release penetrating member having an outer layer comprising a intestine lumen environment resistant component for a period of time while preventing active agent release and/or maintaining physical strength and/or maintaining shape to allow for a controlled penetration of the second state of the penetrating member across or within the small intestine lumen wall said component selected from list comprising a lipophilic or thin film coating, a pore forming component, an interstitial space sensitive biodegradable polymer; and puncturing the small intestine lumen wall with the second state of the penetrating member (e.g., one or a plurality of microneedles), said second state of the penetrating member comprising an active agent release component selected from the group comprising: a biodegradable excipient, a pore forming component, an interstitial space sensitive biodegradable polymer); and delivering the active agent across or within the intestine lumen wall.

In various embodiments of any of the aspects presented, the solid controlled-release penetrating member has a length suitable to reach the interstitial space in the intestine lumen wall.

In some embodiments, the solid controlled-release penetrating member is conical, cylindrical, tubular, pyramid-shaped, cube, pyramid or a hook-shaped. In some examples, the penetrating member is having a pyramid shape. In some embodiments, a penetrating member has a length from base to tip of less than about 1 mm. In some embodiments, the penetrating member has a length of about 20 to 900 μm, 200 to 1000 μm or 500 to 900 μm or 700 to 800 μm. In some embodiments, the greatest radius of the penetrating member is or 100 and 400 μm. In some embodiments, a penetrating member has a maximum diameter or length at the base of the penetrating members between 100 to 1000, or 100 to 600 or 100 to 500 or 100 to 400 or 50 to 1000, or 50 to 600 or 50 to 500 or 50 to 400 μm or 150 to 300 μm.

In some embodiments, the one or more solid controlled-release penetrating members is arranged in an array as a plurality of members, e.g., as microneedles. In some embodiments, array includes about 100-150 penetrating members per cm². In some embodiments, the array or plurality of penetrating members or microneedles are operatively connected to a pliable base. The pliable base may be folded or rolled to be contained in a container for oral delivery. In all embodiments, the pliable base protects the penetrating member from exposure to fluid and no additional layer or coating surrounds the penetrating member on the pliable base end. In some embodiments, the plurality of microneedles is an amount of about 1000 to 2000 or about 1300 to 1500. In some embodiments, the array includes a range of longitudinal sizes ranging between 200-1000 μm. In some embodiments, the penetrating members are spaced more than 0.2 mm or more than 0.5 mm apart from one and other. In some embodiments, the penetrating members are spaced less than 4 mm or less than 3 mm or less than 2 or less than 1 mm apart from one and other. In some embodiments, the penetrating members are spaced about 0.25 to 5 mm or 0.25 to 3 mm or 0.5 to 2 mm apart from one and other.

It should be noted that intestine environment protective component and the degradable component can be embodied by one component with altering characteristics dependent on the environmental conditions. In this case, one component may be triggered to release active agent when certain conditions are met such as pH, viscosity, ion strength, polarity, diffusion, erosion, pressure-based fluid convection or lipophilic conditions, each being specific to the intestine wall tissue. In this case, the distinguishing propertied of the interstitial space compared to the intestinal lumen environment can be used to select a relevant polymer to control the release in the desired target location.

In some embodiments of any of the aspects presented, the array of microneedles is covered as a whole by the intestine environment protective component or controlled release coating.

In some embodiments of any of the aspects presented, the intestine environment protective component is configured to gradually expose the array of penetrating members via biologic, mechanical or chemical degradation (e.g., via dissolving or disintegrating or chemical degradation of excipients). In some embodiment, the intestine environment protective component does not impede exposure of a compartment in a device but rather makes up part of the penetrating member.

The present disclosure provides formulations for one or more penetrating members operably coupled to an intestine wall deployment assembly and uses thereof. In one aspect, the present disclosure provides an oral device for delivery of active agent across or within a lumen of the small intestine of an active agent across or within the small intestine lumen wall in a subject comprising: an intestine release container; and an intestine wall deployment assembly operably connected to one or more intestinal penetrating members described herein.

In some embodiments, the intestine wall deployment assembly is configured to position the solid controlled-release penetrating member adjacent to an intestinal lumen wall and cause the penetrating member to pierce the lumen wall. In one example, an intestine wall deployment assembly is operably coupled to a patch or array of penetrating members and wherein the patch is a gas driven expandable member for patch deployment which is activated by the intestinal liquids to produce a gas such as carbon dioxide (CO₂) or hydrogen gas.

In another aspect, an orally administered drug delivery device is provided that includes a intestine release container and an intestine wall deployment assembly configured for intestine wall deployment and further comprising one or more penetrating members configured for: (i) preventing active agent release and/or maintaining shape and/or strength in the intestine lumen environment for an exposed period of time; and (ii) subsequent release across or within the small intestine lumen wall over a release period of time of the drug during transmucosal penetrating of the penetrating member.

In yet another aspect of the present invention, there is provided an oral device for delivery of active agent across or within a lumen of the small intestine of an active agent across or within the small intestine lumen wall, the formulation comprising: a. an outer intestine release container (e.g., enteric capsule or enteric coated soft gel cap); and b. a drug delivery device or intestine transmucosal patch comprising: i. an expandable member that is associated with one or more penetrating members, said expandable member having proximal and distal surfaces that face in generally opposite directions and configurable in a compacted configuration, an unconstrained configuration, and an expanded configuration. ii. a gas generating compartment in fluid communication with the expandable member and having at least a water or humidity permeable window and configured to expand the pliable expandable member from an unconstrained configuration to an expanded configuration; and iii. one or more penetrating members described herein operably coupled to said pliable expandable member and extending from the proximal surface thereof.

In some embodiments, two or more gas generating compartments are present. In some embodiments, the gas generating compartment is positioned outside and or distinct from the expandable member. In some embodiments, a surface of the wall forming the gas generating compartment is water permeable. In some embodiments, two opposing surfaces of the gas generating compartment wall forming the gas generating compartment is water permeable. In this context, water absorbing may be liquid or gas water absorbing.

In some embodiments, an intestine wall deployment assembly includes i. an expandable member that is associated with one or more solid controlled-release penetrating members, said expandable member having proximal and distal surfaces that face in generally opposite directions and configurable in a compacted configuration, an unconstrained configuration, and an expanded configuration; and ii. a gas generating compartment in fluid communication with the expandable member.

In some embodiments, the solid controlled-release penetrating member are elongated and sized to penetrate a mucosal barrier of the intestinal wall to release an active agent and comprising: an active agent, an intestine environment protective component for maintaining strength and/or shape and/or resisting active agent release during exposure to the intestinal lumen environment for a period of time and an active agent release component for subsequent release of the active agent across of within the small intestine lumen wall for a period of time to release the active agent.

In some embodiments, the expanded shape system is sized and shaped to maintain contact with the intestinal wall (e.g., of the upper small intestine including the duodenum or jejunum or alternatively other sections of the small intestine such as the ileum) by applying an outwardly directed pressure to the intestinal wall to allow for one or more penetrating members to transport at least a portion of the therapeutic agent across the mucosa of the intestine or specifically upper small intestine including the jejunum or duodenum.

In some embodiments, two or more gas generating compartments are present. In some embodiments, the gas generating compartment is positioned outside and or distinct from the expandable member. In some embodiments, a surface of the wall forming the gas generating compartment is water permeable. In some embodiments, two opposing surfaces of the gas generating compartment wall forming the gas generating compartment is water permeable. In this context, water absorbing may be liquid or gas water absorbing.

In yet another aspect, a method is provided for penetrating the mucosal tissue to deliver an active agent comprising: a. converting a pliable expandable member from an unconstrained configuration to an expanded configuration, said expandable member being operably connected to one or more penetrating members in an intestinal protected from comprising a layer or film or pore forming polymer (e.g., a plurality of microneedles) which are exposed to the intestine environment for a period of time; b. biologic (e.g., enzymatic), chemical or mechanical degradation of a layer or film or pore forming polymer to expose an exposed form of the one or more penetrating members; c. penetrating the mucosal barrier with one or more penetrating members (e.g., a plurality of microneedles); and d. releasing active agent over a period of time.

In another aspect, presented is a method of delivering an active agent across or within the small intestine lumen wall, the method comprising the steps of: deploying a drug delivery device (e.g., patch) adjacent to a small intestine lumen wall said drug delivery device comprising one or more penetrating members (e.g., a plurality of microneedles), a pliable base and an expandable member; a. penetrating the mucosal tissue with one or more penetrating members (e.g., a plurality of microneedles) that extend from, or are extendable from, the expandable member at a selected time after the drug delivery device is deployed in the lumen; and b. releasing an active agent in a controlled manner over a period of between 30 minutes and 4 hours such that the active agent is delivered through the plurality of microneedles into the mucosal tissue or is delivered to the region of the mucosal tissue that is disrupted by the microneedles.

In another aspect, a method is provided for delivering an active agent across or within the small intestine lumen wall, the method comprising the steps of a, receiving, by a subject, an oral device for delivery of active agent across or within a lumen of the small intestine comprising i. an outer intestine release container and ii. a drug-delivery device contained within the release component, which includes an active agent formulation shaped as a penetrating member and configured to be advanced across or within the small intestine lumen wall; b. swallowing the drug delivery device by the subject; c. degrading or dissolving the outer intestine release container in the presence of an intestine lumen environmental condition; d. exposing the penetrating members to the intestine lumen environment for a period of time while preventing release of the active agent and/or maintaining strength and/or maintaining shape of the penetrating member to advance the at least one penetrating member across or within the small intestine lumen wall tissue; and e. releasing of the active agent into the tissue of the small intestine over a period of time.

In some embodiments, the choice of excipients involved in degrading or dissolving the container, exposing of the members as well as releasing of the active agent into the tissue of the small intestine are dependent on specific environmental conditions in the lumen, mucosal layers and/or interstitial space. Environmental conditions include but are not limited to pH, ion strength, buffer capacity, or enzymes in the at that point of time.

In some embodiments, strength is measured by applying a constant or limited range of penetration force over area on a defined surface without affecting the shape in any dimension and maintaining strength is less than 5% change in strength at the two timepoints.

In another aspect, there is provided a method for delivering an active agent across or within the small intestine lumen wall, the method comprising the steps of: i. receiving, by a subject, an oral device for delivery of active agent across or within a lumen of the small intestine comprising: (i) an outer intestine release container and (ii) a drug-delivery device contained within the release component, which includes an active agent formulation shaped as a penetrating member for advancing a penetrating member across or within the small intestine lumen wall, the expandable member expanded by gas generating compartment comprising a water or humidity permeable window; configured to penetrate a mucosal barrier of the small intestine lumen wall, an expandable member configurable in a compacted configuration, an unconstrained configuration and an expanded configuration; ii. swallowing the drug delivery device by the subject; iii. degrading or dissolving the outer intestine release container in the presence of an intestinal lumen environmental condition; iv. exposing the penetrating members to the intestine environment for a period of time while preventing release of the active agent and/or maintaining strength and/or maintaining shape of the penetrating member to allow for a period of time for the expandable member to reach an expanded configuration to advance the at least one penetrating member across or within the small intestine lumen wall tissue; and v. release of the active agent across or within the small intestine lumen wall over a period of time.

In yet another aspect of the present invention, there is provided a method for delivering an active agent across or within the small intestine lumen wall, the method comprising the steps of: a. receiving, by a subject, oral device for delivery of active agent across or within a lumen of the small intestine comprising: (i) an outer intestine release container and (ii) a drug-delivery device contained within the release component, which includes (a) an expandable member for advancing a penetrating member across or within the small intestine lumen wall, the expandable member expanded by gas generating compartment comprising a water or humidity permeable window; and a penetrating member; and swallowing the drug delivery device by the subject; b. dissolving the outer intestine release container (e.g., enteric layer as enteric capsule or enteric coated soft gel) in the small intestine; c. exposing a first state of the penetrating member having an outer layer or coating comprising a lumen environment resistant component for a period of time while preventing active agent release and/or maintaining physical strength and/or maintaining shape to allow for a controlled penetration of the second state of the penetrating member across or within the small intestine lumen wall said component selected from list comprising a lipophilic or thin film coating, a pore forming component, an interstitial space sensitive biodegradable polymer; and d. puncturing the small intestine lumen wall with the second state of the penetrating member (e.g., one or a plurality of microneedles), said second state of the penetrating member comprising an active agent release component selected from the group comprising: microspheres, a biodegradable excipient, a pore forming component, an interstitial space sensitive biodegradable polymer); and e. delivering the active agent across or within the intestine lumen wall.

In some embodiments, the active agent release component can include microsphere, one or multiple layers, active agent disposed in a polymer matrix.

There are a number of methods for providing a sustained release film which are then used to coat the solid controlled-release penetrating member. For example, a coating may be prepared by applying and then drying a formulation such as one of the following: i. cellulose ethers such as carboxymethylcellulose, hydroxypropyl cellulose (e.g., Klucel®), hydroxypropyl methylcellulose (e.g., Methocel® E5), ethylcellulose wherein the cellulose ether is in an amount between 1% and 3% w/v, and optionally further comprising a plasticizer in an amount of plasticizer:cellulose ether ratio of 1:10. Relevant plasticizers in this context include but are not limited to PEG (PEG400, PEG2000, PEG6000), glycerol, polypropylene glycol or poloxamer (such as Lutrol F-68 NF). An off the shelf product can also be used such as pseudolatex, known as Surelease®.ii. a hydroxypropyl methylcellulose in an amount of between 2 to 20%. iii. a low-viscosity ethyl cellulose having a viscosity less than about 15 cP and a pore-forming agent selected from the group consisting of hydroxypropyl cellulose and hydroxypropyl methylcellulose; and iv. polyvinyl pyrrolidone (for example, PVP k-25-PVP k-360) in a concentration between 5% and 30% or preferably between 1 and 10%, depending on the grade and molecular weight, as would be recognized by those familiar in the art.

### BRIEF DESCRIPTION OF DRAWINGS

The drawings are for illustration purposes only and should not introduce limitation.

FIG. 1 are schematic diagrams of the longitudinal sectional view of solid controlled-release penetrating members, according to one set of embodiments having a cylindrical shape according to the present invention.

FIG. 2 are additional schematic diagrams of the longitudinal sectional view of solid controlled-release penetrating member s, according to an additional set of embodiments having a cylindrical shape according one set of embodiments of the present invention.

FIG. 3 are schematic diagrams of the transverse sectional view of solid controlled-release penetrating members, according to an additional set of embodiments having a cylindrical shape according one set of embodiments of the present invention.

FIGS. 4A and 4B are schematic diagrams of the oral device for delivery according one set of embodiments of the present invention.

### DESCRIPTION OF EMBODIMENTS

For the present disclosure to be more readily understood, certain terms are defined below. Additional definitions for the following terms and other terms are set forth throughout the specification.

As used herein, “drug”, “active agent”, “therapeutic” or “medicine” may be used interchangeably and includes small molecules, biologics, diagnostics, or active pharmaceutical ingredients used to treat or diagnose a patient. They can refer to any agent that, when administered, has a therapeutic effect and/or elicits a desired biological and/or pharmacological effect. Generally, an active agent may refer to more than one active agent. Examples of active agents that may be suitable for use include but are not limited to chemotherapeutic agent, interferon, an antibody, an antibiotic, growth hormone, parathyroid hormone, a glucose regulating agent, an insulin compound, an incretin hormone, a GLP-1 compound or exenatide, antiviral, protease inhibitor or an anti-seizure compound or other active agents which would otherwise chemically degrade or have limited permeability if released within the intestine lumen of the gastrointestinal tract. In various embodiments of any of the aspects presented, the active agent is a peptide sequence, protein, an enzyme, a polysaccharide, or a polynucleotide, amino acids, nucleotides, carbohydrates, sugars, lipids, nucleoproteins, glycoproteins, lipoproteins, steroids, etc. whether naturally occurring or artificially created (e.g., by synthetic or recombinant methods) that are commonly found in cells and tissues. Specific classes of biologics include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. In some embodiments of any of the aspects presented, the active agent is in an amount of the active agent to produce a desired therapeutic effect is less than an amount to produce a corresponding effect if the agent was orally delivered without enclosure in the ingestible formulation. In some embodiments of any of the aspects presented, the active agent is a combination of active agents. In some embodiments of any of the aspects presented, the active agent would chemically degrade, have limited permeability, or impose a deleterious effect on the subject if released within the lumen of the gastrointestinal tract. In some embodiments of any of the aspects presented, the active agent comprises a polypeptide that is chemically degraded or have limited permeability in the GI tract and the agent is delivered into the wall of the small intestine with minimal or no loss in binding affinity or specificity to a target binding site.

As used herein, a percentage of penetrating members can be an estimated based on examination of a number of sample size. It is understood that the accuracy of this method may be improved as one increased that number of samples used in any case.

As used herein, degrading includes but is not limited to biodegradation, dissolving or disintegration due to exposure to biological fluid.

As used herein, the intestinal wall for deployment of the device is typically the upper small intestine including but not limited to the duodenum or jejunum but may also include the ileum.

As used herein, the intestine lumen environment is the environment typical of the lumen of a human intestine.

As used herein, “coating” may refer to complete or partial covering. The coat may be continuous, discontinuous, flat, or textured. They may be smooth or follow contours of pores that may be present on the surface which they are covering.

As used herein, pore-forming polymers are biocompatible and readily water-soluble such as hydroxypropyl cellulose, hydroxypropyl methylcellulose, polyethylene glycol (PEG), polylactic (also termed polylactide), polyglycolic acid (also termed polyglycolide), a polylactic-polyglycolic acid copolymer, and polyvinyl alcohol.

As used herein, “microneedle” refers to a type of penetrating member which is typically a protrusion from the surface of a rigid or flexible base. Microneedles generally are shape objects and in theory can be of any shape or design as long as the microneedles are shaped to penetrate or pierce tissue and, in this case, intestinal tissue. A microneedle may be conical, cylindrical, tubular, pyramid-shaped, cube, pyramid or a hook-shaped. A microneedle may be straight, curved, or semi hook shaped. Without being bound to any particular theory, a curved microneedle can facilitate retention of a device disclosed herein at a target site. A microneedle may protrude at angle from a device surface, the microneedle having a base integrally connected to the surface, a tip distal to the base, and a body therebetween. A microneedle or a portion of a microneedle can be porous or non-porous. A microneedle or a portion of a microneedle is biodegradable in the intestinal tissue over a period of time. A plurality of microneedles used in accordance with the present disclosure may include a mixture of different microneedles. For instance, microneedles of the plurality may include microneedles having various lengths, base portion materials, body portion diameters (i.e., gauge), tip portion shapes, spacing between microneedles, coatings, etc.

Note that Eudragit® copolymers are commercialized by Evonik and their composition is known to the skilled artisan

Although intestinal wall is used throughout, there are some cases where the invention may equally apply to similar soft tissue.

The present invention will be more fully understood from the following detailed description of embodiments thereof, taken together with the drawings.

Various embodiments of the invention can be used to deliver active agents or active pharmaceutical ingredients (“API”) that were previously only delivered by parenteral administration. In various embodiments, such therapeutic agents may include biotherapeutic agents (also described as biologics) or peptides.

An intestinal transmucosal drug delivery device is provided for deployment or placement at the intestinal lumen wall after oral administration or due to surgical placement and generally maintained for the duration of delivery of an active agent within or to the lumen wall so long as anchorage in the wall is maintained. Key to successful delivery of an active agent without early release into the GI tract where it made degrade is the penetrating member. This one component is required to encompass many conflicting goals including: maintenance of shape and structure without early release of active agent during exposure to intestinal lumen conditions (which include both the fluid in the lumen and mucosal tissue) such that penetration is effective during a deployment at the lumen wall; subsequent release of active agent once positioned in the intestine lumen wall while maintaining anchorage in tissue; subsequent biodegradation.

A transmucosal drug delivery device is provided for deployment in the lumen of the intestine, preferably at the upper small intestine (including but not limited to the duodenum or jejunum). The deployment, or placement, of the device as well as the mechanism by which to initiate penetration of the penetration member can be by any method known in the art including that disclosed in WO2020035857.

### Penetrating Member

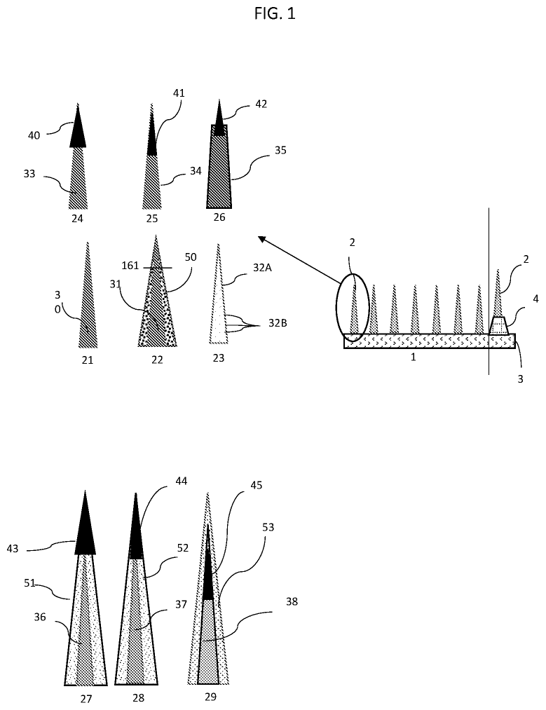

A number of embodiments of penetrating members suitable for use in accordance with the present invention are illustrated in FIGS. 1-3.

FIG. 1-3 are schematic diagrams of the longitudinal and transverse sectional view of solid controlled-release penetrating member s, according to one set of embodiments.

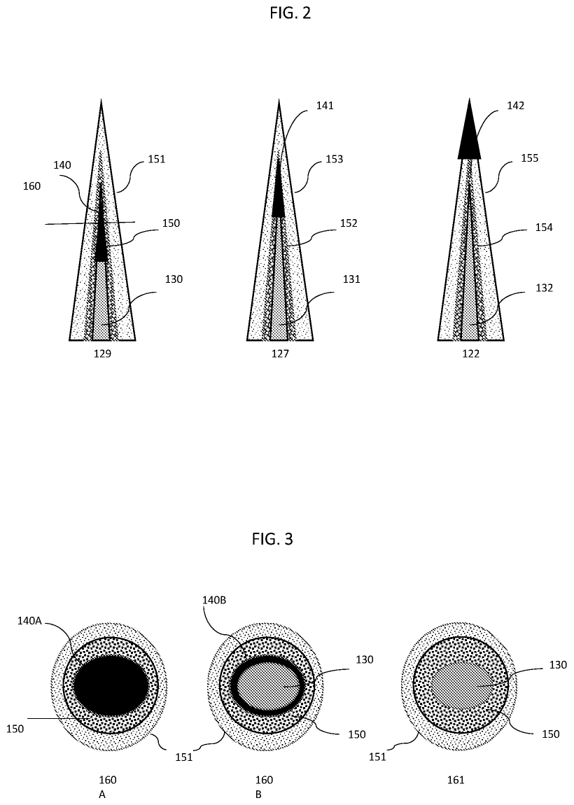

In one embodiment, a number of solid controlled-release penetrating members 2 is arranged on a pliable member 3 to form an array 1 of penetrating members 2. In some cases, the penetrating member 2 may have geometric properties or a harder material forming an additional component 4 at the base of the penetrating member to enhance penetration efficacy, especially important in polymeric based penetration members. The longitudinal sectional view of penetrating members 21-29 and 122, 127 and 129 according to the present invention are presented in FIGS. 1 and 2. Note that the penetrating members are drawn as having cylindrical shape, although conical, cylindrical, tubular, pyramid, cube, pyramid-shaped or a hook-shaped are also envisioned. Note as well that the layers are not drawn to scale and do not represent any relative measurements. In all penetrating members in FIGS. 1 and 2, during exposure to the intestinal lumen environment which we will define to include the mucous in the target wall, geometric and morphological qualities are maintained. For example, there may be less than 10% change in size of the penetrating member in any dimension at 10 minutes, during (or after) an intestine lumen exposed period or less than 10% release of active agent at 10 minutes. Alternatively, some embodiments of penetrating members in FIGS. 1 and 2, during exposure to the intestinal lumen environment, the tip in substantially maintained. In this context, more than 12 μm diameter or length, more than 10 μm diameter or length or more than 8 μm diameter or length of the tip is maintained wherein the penetrating member is a cone, pyramid or other shape. In addition, the timing for release of active agent is between 15 minutes to 120 minutes meaning that the desired release rate of the active agent is obtained despite the resistance to the environment.

In various embodiments, the solid controlled-release penetrating member includes an active agent-controlled release formulation located in the center and/or protected by outer layers or coatings. In the various embodiments described in the following figures, the active agent an active agent which chemically degrades, is poorly absorbed, and/or is not well tolerated in the GI tract. In one embodiment, such as solid controlled-release penetrating member 21, the active agent formulation 30 is a mixture or matrix with an excipient such as a polymer. In various other embodiments such as the solid controlled-release penetrating member 22, the active agent formulation 31 is an active agent inner core and the controlled release component is present as at least a partial controlled release coating 50 on the active agent inner core to form a controlled release coating. In various other embodiments, the solid controlled-release penetrating member 23 includes a molding or polymer material 32A comprising active agent loaded particles 32B such as rigid particles or microparticles dispersed within the molding material, wherein the molding material is shaped or molded (e.g., micromolded) into a penetrating member. The choice of molding material and or excipient or polymer are chosen such that the active agent activity is preserved during the manufacturing process, during shelf life of the product to maintain stability and for optimized environment created during release.

In various embodiments, the solid controlled-release penetrating member shown in 24, 25 and 26 includes a tip shaped to promote penetration. For example, the tip may have an angle of between 20 and 40 degrees, or about 30 degrees. It will be recognized that the larger the surface of the tip (i.e., the surface which is positioned to penetrate tissue first), the greater the force required for effective penetration. The solid controlled-release penetrating member shown in 24, 25 and 26 have intestine environment resistance to degradation such that during exposure to the intestinal lumen environment, the penetrating member or at least 80% or the array of penetrating members undergoes less than 10% change in size of penetrating member in any dimension at 10 minutes during an intestine lumen exposed period or less than 10% release of active agent at 10 minutes. Note that when the penetrating member is a microneedle, the tip may be especially prone to breakage. Thus, in some embodiments, the tip is enforced as exemplified the additional tip coating 40 located on the distal end of penetrating member 24 and as a partial external coating on the distal end of active formulation 33. In another example, penetrating member 25 includes an active agent formulation 34 (in this case active agent loaded microparticles contained or shaped as a penetrating member), having an adjacent and distal extension of the controlled release formulation to form an intestine resistant tip 41, for example as can be prepare by adding a tip formulation to a mold, centrifuging the formulating in mold and drying to form a tip layer or coating, and subsequently, adding a second formulation containing the active agent to fill the remaining volume of the mold. In this case, the tip is formulated as a distinct layer or coating which is slow dissolving at the tip 41 or 42 and prepared as part of a bilayer penetrating member. In this case, during exposure to the intestinal lumen, there is less than 10% change in size of the penetrating member or at least 80% of the array of penetrating members in any dimension at 10 minutes during an intestine lumen exposed period or less than 10% release of active agent at 10 minutes. This approach may also include alternative shapes of molds including penetrating member 26, having tip 42 and active agent formulation 35. Thus, there are two methods, a tip coating approach which surrounds at least of portion of the tip of the active agent formulation to form a penetrating member or an adjacent formulation which forms the tip itself and resides adjacent to the active agent formulation. Both may be effective in achieving the structural integrity of the tip required for subsequent penetrating in the soft tissue of the intestine after exposure to the intestine lumen environment for an exposure period. Note that a coating or adjacent tip may be used on any active agent formulation—whether the active agent formulation is exemplified by any of 21, 22 or 23.

In additional embodiments such as penetrating member 29, an intestine environment protective component or controlled release coating 52 may surround the previous layers including the active agent formulation 38 and the tip 45 (which may be formulated as a coating as described in 40 or adjacent layer as described for tip 41), such that the intestine environment protective component or controlled release coating 52 ensures that degradation resulting from exposure to the intestinal lumen environment, does not affect the ability of the tip to subsequently penetrate the intestinal tissue and reach the target layer, the interstitial space where drug can be released and reach the blood or lymphatic system.

In alternative embodiments, such as penetrating member 27 and 28, an additional coating or layer of controlled release coating or intestine environment protective component 51 or 52, may be present under or adjacent to a tip formulation. Coating 51 and 52 may be a sustained release polymer or pH sensitive or cationic acrylic/methacrylic copolymer. For example, a film-forming polymeric material such an enteric acrylic/methacrylic copolymer which has a pH threshold of above 6.5 or about 7.0 or above may be used. In some embodiments, the tip formulation 43, 44 and 45 may be formulated as slow dissolving at the tip and prepared as part of a bilayer penetrating member or alternatively as a tip coating and may be a coating at least partially surrounding the intestine environment protective component which at least partially surrounds the active agent formulation 36. Alternatively, the active agent formulation 37 may have an intestine environment protective component 52 at least partially surrounding the active agent formulation and further including an adjacent formulation which forms tip 44, at the distal end of penetrating member 28. In these embodiments, the active agent formulation 36, 37 and 38 may be a mixture or matrix with an excipient such as a polymer as exemplified in 21, an active agent formulation having an active agent inner core and the controlled release component is present as at least a partial coating on the active agent inner core to form a controlled release coating, as exemplified in 22 or alternatively, as active agent loaded rigid particles such as microparticles dispersed within the molding material as exemplified in 23.

In some embodiments displayed in FIG. 2, in penetrating members 129, 127 and 122, there is a controlled release component 150, 152, and 154 as well as an additional layer or coating being an intestine environment protective component 151, 153 and 155. In this case, the active agent formulation 130, 131 and 132 may be a mixture or matrix with excipients or active agent loaded rigid particles (e.g., microparticles) 51 or 52. In one embodiment such as penetrating member 129, the tip may be a coating surrounding the active agent formulations or alternatively, as a separate component adjacent to and at a distal end of the active agent formulation to form a tip 140 and further coated by a controlled release component and an intestine environment protective component 151. In another embodiment, such as penetrating member 127, the tip may be a coating surrounding the controlled release component 152 or alternatively, as a separate component adjacent to and at a distal end of the controlled release component 152 which surrounds an active agent formulation 131 to form a tip 141. In this case, tip 141 is further surrounded by an intestine environment protective component 153. In yet another embodiment, tip 142 is a coating surrounding the intestine environment protective component 155 or alternatively, as a separate component adjacent to and at a distal end of the intestine environment protective component 155.

In some embodiments displayed in FIG. 3, a cross sectional view of penetrating member 129 is displayed in order to exemplify the different options for the tip in all of the embodiments. 161 is a cross sectional of penetrating member 21 which includes no tip reinforcement but additionally includes an intestine environment protective component not portrayed in penetrating member 21. The active agent formulation 130 is in the center and protected from early release due to exposure to the intestine environment and is surrounded by controlled release coating 150 and an intestine environment protective component 151 which provides a delayed but controlled release of active agent of between 5 minutes to 4 hours, or preferably between 15 minutes and 120 minutes. In another embodiment, 160A and 160B, the tip is reinforced to ensure effective penetration after exposure to the intestine environment. The tip may be a coating 140B surrounding the active agent formulation 130 and further coated by a controlled release coating and/or intestine environment protective component or alternatively, the layers or order of coatings may be switched, and the tip coating may be surrounding the controlled release coating and under the intestine environment protective component. In another embodiment, the tip 140A is a separate component adjacent to and at a distal end of the active agent formulation and coated by a controlled release formulation 150 and/or an intestine environment protective component 151.

Any of the above described embodiments, ensures that there is substantially no degradation nor change in shape or size of the penetrating member resulting from exposure to the intestinal lumen environment such that there is little to no effect on the ability of the tip to subsequently penetrate the intestinal tissue and reach the target layer, the interstitial space, where the active agent can be released from the penetrating member and reach the blood within less than the desired time and especially before release of the intestine wall deployment assembly disengages from the wall.

In some embodiments of any of the aspects presented, the one or more penetrating members comprise an active agent and an enteric polymer having a threshold or sensitivity to degradation at a pH greater than 6.5 or about 7.0. In some embodiments, the enteric polymer forms at least a partial layer around a formulated active agent. In one example, a methyl acrylate methacrylic acid copolymer such as Eudragit® enteric polymer or more specifically a methyl acrylate-methyl methacrylate-methacrylic acid copolymer such as Eudragit® S or Eudragit® L30D55 can be used. It should be recognized to those in the art that during the coating process and curing temperature, the stability of the active agent should be preserved. It should be recognized to those in the art that these copolymers can be used alone or with a plasticizer. Such layers are normally applied using a liquid medium, and the nature of the plasticizer depends upon whether the medium is aqueous or non-aqueous. Plasticizers for use with aqueous medium include propylene glycol, triethyl citrate, acetyl triethyl citrate or Citroflex® or Citroflex® A2. Non-aqueous plasticizers include these, and diethyl and dibutyl phthalate and dibutyl sebacate. A preferred plasticizer is Triethyl citrate.

In some embodiments, two or more layers are provided. In one example, the one or more penetrating members comprise: at least a partial layer or coating comprising an enteric polymer which selectively degrades in the intestine wall (e.g., having a pH threshold greater than 6.5 or about 7.0); and at least a partial external coating of an intestine resistant component on the penetrating member or a tip thereof (lipophilic coating).

In another embodiment, two or more layers are provided such that the penetrating members comprise: i. at least a partial layer or coating comprising an enteric polymer which selectively degrades in the intestine wall (e.g., having a pH threshold greater than 6.5 or about 7.0); and ii. at least a tip coating of a lipophilic or water insoluble excipient.

In another embodiment, three layers or coatings are provided such that the penetrating members comprise: i. at least a partial layer or coating comprising an enteric polymer which selectively degrades in the intestine wall (e.g., having a pH threshold greater than 6.5 or about 7.0); and ii. at least a tip coating of a lipophilic or water insoluble excipient; and iii. at least a partial layer or coating comprising an enteric polymer which selectively degrades in the intestine wall (e.g., having a pH threshold greater than 6.5 or about 7.0); and at least a partial external coating of an intestine resistant component on the penetrating member or a tip thereof (lipophilic coating).

### Methods for Fabrication

A description is now provided for the fabrication process used to make various embodiments of the solid controlled-release penetrating member of the present invention. Methods for fabricating the solid controlled-release penetrating member including methods for fabricating a plurality of penetrating members and especially an array of penetrating members operably coupled to a pliable base are provided. The process and formulation were designed to meet challenges presented by the sensitivity to degradation of the preferred active agents, peptides, and proteins. In addition, the process and formulation support the operable connection of the penetrating members to a pliable base.

For preparing an array of penetrating members operably connected to a pliable base, a 3M mold can be used to produce a PDMS mold. Optionally, if a tip layer or coating is desired, a formulation for the tip can be poured prior to the active agent and centrifuged for 30 min for concentration in the tip. The active agent is then prepared with relevant excipients which are relevant to preserve activity and placed or poured into the mold. The mold is centrifuged to remove excess and then a pliable base formulation such as PVP solution can be applied on the mold and centrifuged and dried under room temperature conditions.

Once the array of penetrating members or microneedles are prepared, layers or coatings may be applied onto the core by any suitable means known to a person skilled in the art. For example, it can be applied using classical coating or dipping techniques to add a tip coating, control release coating or intestine environment protective component. Some selection in methods is necessary in order that the pliable base is not affected in terms of function.

In the masked dip coating or protective masking, for example, an array of penetrating members is dipping into the coating liquid and plate masking is used to avoid coating the pliable base. Alternatively, a thin film dip coating method can be used where a thin coating liquid film is applied by rolling using a doctor blade C-ii, once again with control of the height of the coating and preventing the coating of the pliable base be keeping the height lower than the height of the penetrating member. As a result, when the microneedles are dipped into the thin liquid film, the coating liquid is unable to rise significantly and thus is unable to touch the base.

An additional option may be using spray coating or frozen spray coating. The solvent-based solution of coating is applied and when the target weight gain is reached, the formulation can be dried, and a further coating can be applied. Multiple coatings can thus be applied. Note that the speed of the member exiting the solution, coating solution viscosity, drying time and presence of surfactant between dips contribute to uniform coating.

An additional method may include powder layering where a dry formulation or polymer using rotor dry layering is applied and a subsequent liquid aqueous binder is applied.

In all stages of the process, drying temperatures remain under 40 degrees Celsius, optionally under vacuum conditions. Exact ratios of excipients may be varied as well as coating % and in vitro release measured for further optimization.

### Intestine Wall Deployment Assembly

An exemplary intestine wall deployment assembly suitable for use in accordance with the present disclosure is illustrated in where oral drug delivery device 200 is configured for ingestion by a subject. Although the following intestine wall deployment assembly will now be described in detail, any intestine wall deployment assembly known in the art may be suitable as long as it is capable of positioning the penetrating tip at the inner wall of the small intestine and applying force to the base of the penetrating member. The oral device 200 comprises an intestine release container 201 for example, an enteric capsule or alternatively present is a core which is directly coated with enteric coating.

The intestine release container 201, is configured to dissolve in the small intestine environment 202 contained within the small intestine walls 203 (e.g., a duodenum, jejunum, and/or ileum walls) and resist degradation in the gastric environment and until arriving in the lumen of the small intestine 210 of the subject. Typically, intestine release container 201 is pH-sensitive and may be configured to dissolve within 15 minutes (e.g., within 10 or 5 minutes). Typically, the intestine release container 201 has a length of at least 5 mm, no more than 30 mm, and/or between 5- and 30-mm dissolving. Typically, the intestine release container 201 has a diameter of between 3 and 6 mm or between 4 and 5 mm.

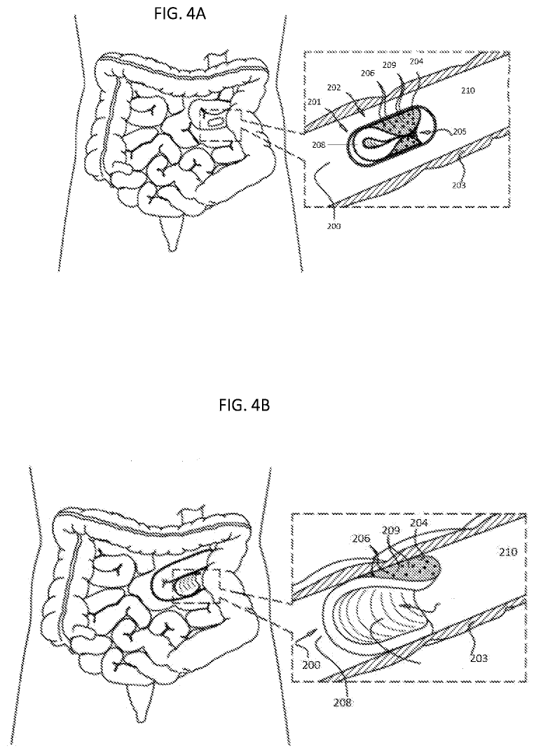

The oral drug delivery device 200 in FIG. 4A and FIG. 4B may comprises (a) an expandable member 208 having a proximal (intestinal-wall-contact) surface 204 and a distal (intestinal-lumen-facing) surface 205, which face in generally opposite directions, (b) one or more penetrating members (e.g., microneedles) 206 as described above and operably connected to a pliable base 209, and (c) a gas generating compartment having a water or humidity permeable window. In some embodiments, two or more gas generating compartments are present. In some embodiments, the gas generating compartment is positioned outside and or distinct from the expandable member. In some embodiments, a surface of the wall forming the gas generating compartment is water permeable. In some embodiments, two opposing surfaces of the gas generating compartment wall forming the gas generating compartment is water permeable. In this context, water absorbing may be liquid or gas water absorbing.

The oral drug delivery device 200(a) has a compressed shape when disposed within intestine release container 201, as shown in FIG. 4A, and (b) is configured to assume, after release from the intestine release container 201 (when it at least partially degrades, disintegrates or dissolves), an unconstrained shape and subsequently assume an expanded shape as shown in FIG. 4B, in which an expandable member 208 has an outer perimeter. FIG. 4B shows the drug delivery device 200 in expanded shape, adjacent to a wall of small intestine 203 in which penetrating members 206 are exposed for a period of time to the small intestine environment 210 during transition from the unconstrained shape, immediately after the intestine release container has at least partially degraded or dissolved, but prior to the fully expanded form wherein the penetrating members are forced into the wall of the small intestine 203. Even in the fully expanded form of the oral drug delivery device 200, the fully inner perimeter of the small intestine is not in contact with the expandable member 208 (i.e., it does not encompass the full inner volume of the lumen at any specific location), such that food and liquids may pass via 211. Typically, an expandable member 208 has elastomeric character and comprises a non-metal material, such as a plastic material (e.g., thermoplastic polyurethane or silicone). The drug delivery device 200 expands, such as by unfolding, and/or unrolling, in response to no longer being constrained by intestine release container, and/or in response to contact of drug delivery device 200 with fluid in small intestine environment 210. Preferably, the surface of the expandable member 204 does not stretch and forms a substantially flat surface such that it has a variance of height of about 5 mm or 4 mm or 3 mm or 2 mm.

For some applications, drug delivery device 200 is biodegradable along the gastrointestinal tract. For some applications, an expandable member 208 comprises an elastomer, such as polyethylene or silicone which does not biodegrade but rather passes safely through the gastrointestinal tract without sharp or rigid elements. For some applications, there are no sharp or rigid parts of the drug delivery device except for biodegradable penetrating members (microneedles).

Typically, an expandable member 208 is configured such that expanded shape in FIG. 4B is generally flat when drug delivery device 200 is expanded after degradation of the intestine-release container 201. For some applications, a gas motor is configured to be initiated by complete or partial degradation of the intestine-release container (e.g., enteric layer) 201, which subsequently expands the unconstrained expanding member to assume an expanded configuration as an expanded expandable member which assumes a curved shape when constrained by wall 203 of small intestine, such as shown in FIG. 4B and is then positioned to advance the one or more penetrating members 206 into the wall of the small intestine. Typically, a gas-generating formulation that produces gas upon contact with liquid or humidity is disposed within a compartment.

The expanded expandable member 208 has proximal surface 204 of an expandable member which eventually contacts the intestinal wall 203 in expanded form, thereby bringing one or more penetrating members 206 into contact with intestinal wall 203. Typically, one or more penetrating members 206 penetrate intestinal wall 203, to release the active agent without inducing release in the lumen. In addition, one or more expandable members 208 may serve as a temporary anchor that holds the drug delivery device in place during active agent delivery. Once expanded, proximal surface 204 of an expandable member 208 establishes good (complete or nearly complete) contact with intestinal wall 203, as shown in FIG. 4B. The shape and dimensions of expandable member 208 may contribute to this good contact by preventing other, lower-contact-level orientations of the expandable member in the lumen of small intestine 203. Drug delivery device 200 and its associated small intestine tissue 203 typically remain axially stationary in the small intestine for a release period to enable release of an active agent while the penetrating members are within the tissue of the intestinal wall 203, even under contraction of the small intestine wall 203. Following delivery of the active agent, the drug delivery device 200 continues through the GI and is eventually passed from the body. The location of an expandable member 208 is generally unaffected by peristalsis and does not block liquids or food passing through the intestine.

Generally, the outer intestine release container may be sized and shaped to be swallowed and pass into the gastrointestinal tract with release in the small intestine specifically. Enteric formulations for capsules for example are known in the art. The capsule container is necessarily configured to resist degradation in the stomach and degrade in whole or in part during passage in the small intestine. In some variations, the material of the capsule container may be configured to degrade in an intestinal environment (e.g., in the small intestine) in which the pH is at least about 5.5. For example, the capsule container may be formed from a material and/or include a coating that is configured to degrade in an environment having a pH of at least 5.5, at least 6.0, at least 6.5, at least 7.0, at least 7.1.

The capsule container may be configured to dissolve in its entirety, and/or the capsule container may break apart into smaller pieces (e.g., due to dissolvable joints or seams) to facilitate release of the delivery device into the small intestine for deployment.

The outer intestine release container can be an enteric coating or layer surrounding the drug delivery device (e.g., an enteric capsule) or alternatively an enteric coated soft gel cap. The term enteric is generally used to describe materials that are stable at relatively highly acidic pH conditions (e.g., pH of less than about 5.5) and susceptible to dissolution at relatively alkaline pH conditions (e.g., pH of between about 6 and about 9). In some embodiments, the enteric polymer includes, but is not limited to, enteric polymer can be selected from the group consisting of poly(methacrylic acid-co-ethyl acrylate), cellulose acetate phthalate, cellulose acetate succinate, hydroxypropyl methylcellulose phthalate, methylcellulose phthalate, ethylhydroxycellulose phthalate, polyvinyl acetate phthalate, polyvinyl butyrate acetate, vinyl acetate-maleic anhydride copolymer, styrene-maleic mono-ester copolymer, methacrylic acid methyl methacrylate copolymer, methyl acrylate-methacrylic acid copolymer, methacrylate-methacrylic acid-octyl acrylate copolymer; and copolymers, mixtures, blends and combinations thereof. Hydroxypropyl methylcellulose acetate succinate (HPMCAS) is a particularly useful enteric polymer, and can be used alone or in a copolymer, mixture, blend, or combination with any one or more of the other foregoing enteric polymers.

In some embodiments, the wherein the outer intestine release container at least partially or fully contains an array of penetrating members as well as the intestine wall deployment assembly.

In general, devices described herein can comprise an expanding member coupled to the penetrating members for advancing the penetrating member into the wall of the small intestine. In some embodiments, the expandable member has proximal and distal surfaces that face in generally opposite directions. In some embodiments, the one or more penetrating members (e.g., microneedle array) extend from the proximal surface thereof.

The expanding and pliable member can be configurable in a compacted configuration, an unconstrained configuration, and an expanded configuration. In some embodiments, the expanding and pliable member is configured to be initiated by exposure to the intestinal lumen environment. In some embodiments of the present invention, the expanding and pliable member can be configurable in a compacted configuration where it is retained within the capsule in a folded state. For examples, the expanding and pliable member can be folded to define one or more creases, which define respective inner and outer crease sides, wherein at least one of the tissues penetrating members is coupled to the patch along the inner crease sides.

In some embodiments, the pliable member can inscribe a circle having a diameter of between 2 and 10 cm when the patch is unconstrained. In some embodiments, the pliable member can inscribe a circle having a diameter of less than 8 cm, less than 6 cm, less than 5 cm minutes or about 4 cm. Combinations of the above-referenced ranges are also possible (e.g., between 2 and 5 cm).

In some embodiments, the expanded pliable member, when deployed in the lumen of the intestine without intestinal peristalsis, applies no force to facing intestinal walls along axial extents thereof. Unlike a balloon based expanded member which expands such that there is contact with most of the surfaces of the intestine wall, the expanded pliable member in this case is in surface contact in only one area of the circumference of the intestine lumen.

In some embodiments, the upper surface of the expandable member contacts an intestinal wall, thereby bringing the penetrating members into contact with the intestinal wall. In some embodiments at least 50% or at least 75% or at least 80% or at least 90% of the penetrating members are coupled to the proximal surface of the patch. In another embodiment, at least 50% or at least 75% or at least 80% or at least 90% of the area of the upper surface of the expandable member is in direct contact with the intestine tissue.

In some embodiments, the expandable member gradually moves from an unconstrained configuration to an expanded configuration due to gas pressure. The gas pressure resulting from the gas generating reaction is sufficient to advance the penetrating member into the wall of the small intestine.

Gas motor can be coupled to the release element and can be configured to be initiated by release of the release element which subsequently causes expansion of the expanding member for advancing the penetrating member into the wall of the small intestine. The gas generating compartment or compartments can be defined by i. a substantially water-permeable and substantially gas-impermeable, polymer layer shaped to define at least one window therethrough, and a liquid and humidity permeable and substantially gas-impermeable wetting layer, which entirely covers the at least one window and is sealed to the polymer layer around the at least one window; and ii. a gas-generating formulation that produces gas upon contact with liquid or humidity, the gas-generating formulation disposed within one or more compartments. The gas generating formulation can include sodium bicarbonate and is typically disposed in a vicinity of the at least one window which permeable to humidity or water.

In an embodiment, techniques and apparatus described in one or more of the following applications are combined with techniques and apparatus described herein: U.S. Pat. No. 8,287,902 to Gross; and/or U.S. Pat. No. 9,492,396 to Gross.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A solid controlled-release penetrating member for use in delivery of active agent to an intestine wall after a period of exposure to the GI track comprising an active agent, a base at a proximal end and a penetrating tip at a distal end thereof, said tip shaped and formulated to promote penetration of the intestine tissue during exposure to an intestine lumen environment (e.g., intestine fluid and/or mucous) wherein when the penetrating member is subject to simulating intestine conditions such as in vitro dissolution testing employing a USP II apparatus with dissolution medium at 370 C, 10-30 mM or preferably 30 mM buffer, pH 6.5 with fasted state simulating intestinal fluid (FaSSIF):
A. at 10 minutes,
i. the penetrating member or more than 80% of the penetrating member array loses less than 20% or 10% of its length,
ii. the penetrating member or array of penetrating members release less than 10% of its active agent or
iii. the penetrating member or more than 80% of the array of penetrating members maintains a diameter of tip of less than 30 μm or 25 μm; and
B. at 120 minutes, the penetrating member releases at least 80% of its active agent.

2. The penetrating member of any previous or subsequent claim, wherein, the penetrating member comprises a portion of a therapeutically effective dose of at least one therapeutic agent.

3. The penetrating member of any previous or subsequent claim, wherein the penetrating member comprises an outer coating of an intestine environment protective component.

4. The penetrating member of any previous or subsequent claim, wherein the intestine environment protective component surrounds a controlled release formulation.

5. The penetrating member of any previous or subsequent claim, wherein the tip is at least partially coated, or the tip is formulated as a separate but adjacent and distal extension of an active agent formulation.

6. The penetrating member of any previous or subsequent claim, wherein the penetrating member has a length of about 200 to 1000 μm.

7. The penetrating member of claim 6, wherein the penetrating member has a length of about 500 to 850 μm.

8. A penetrating member for delivery of an active agent across or within a lumen wall of the small intestine of a subject, said penetrating member sized to be contained within an oral swallowable container and being with a tissue penetrating tip at a distal end and comprising:
a controlled release formulation comprising an active agent which degrades, is poorly absorbed, and/or is not well tolerated in the lumen of the GI tract and a controlled release component; and
an intestine environment protective component at least partially surrounding the controlled release formulation and comprising a lipophilic coating or a film forming polymeric material having a pH threshold at about 6.5 or above.

9. The penetrating member of any previous or subsequent claim, wherein the penetrating member releases the active agent between 15 min to two hours after exposure of penetrating member to intestine environment.

10. The penetrating member of any previous or subsequent claim, wherein the intestine environment protective component is a film-forming polymeric material having a pH threshold at about 6.5 or above.

11. The penetrating member of any previous or subsequent claim, wherein the length of solid tissue penetrating member is less than 1000 μm.

12. The penetrating member of any previous or subsequent claim, wherein the length of solid tissue penetrating member is about 200 to 1000 μm or 500 to 850 μm.

13. The penetrating member of any previous or subsequent claim, wherein, wherein when the penetrating member is subject to in vitro dissolution testing employing a USP II apparatus with 30 dissolution medium at 370 C, 30 mM buffer, pH 6.5 with fasted state simulating intestinal fluid (FaSSIF), the active agent is released from the penetrating member at a rate such that: at 10 minutes, substantially no active agent is released.

14. The penetrating member of any previous or subsequent claim, wherein, when the penetrating member is subject to in vitro dissolution testing employing a USP II apparatus with dissolution medium at 370 C, 30 mM buffer, pH 6.5 with fasted state simulating intestinal fluid (FaSSIF), the active agent is released from the penetrating member at a rate such that, at 10 minutes, there is less than 20% or 10% or 5% or 3% or 1% change compared to the starting size of any dimension.

15. The penetrating member of any previous or subsequent claim, wherein the active agent is chemically degradable, poorly absorbed, or is not well tolerated in the lumen of the gastrointestinal tract.

16. The penetrating member of any previous or subsequent claim, wherein the active agent is a peptide sequence, protein, an enzyme, a polysaccharide, or a polynucleotide.

17. The penetrating member of any previous or subsequent claim, wherein the penetrating member is an array or a plurality of microneedles.

18. The penetrating member of any previous or subsequent claim, wherein the active agent is in an amount of more than 2 mg, more than 4 mg, more than 6 mg, more than 8 mg or in an amount of between 2 and 10 mg in the array.

19. The penetrating member of any previous or subsequent claim, wherein the penetrating member comprises an active agent contained or shaped as a microneedle.

20. The penetrating member of any previous or subsequent claim, wherein the tissue penetrating shape includes a tip or sharp edge for penetrating soft tissue.

21. The penetrating member of any previous or subsequent claim, wherein the tissue penetrating shape has mechanical strength provided by at least a partial tip coating or formulated as a distinct section at the distal end of the penetrating member and optionally comprising any one or more of the following: water insoluble, hydrophobic or non-degradable polymers.

22. The penetrating member of any previous or subsequent claim, which does not include a disintegrant as an excipient.

23. The penetrating member of any previous or subsequent claim, wherein the maximum diameter or length at the base of the tissue penetrating members is between 50 and 500 μm.

24. The penetrating member of any previous or subsequent claim, wherein the penetrating member has a conical, cylindrical, tubular, pyramidal, cube, or a hook shape.

25. The penetrating member of any previous or subsequent claim, wherein the penetrating member is conical, or pyramid shaped.

26. The penetrating member of any previous or subsequent claim, wherein the penetrating member is packaged within an intestine release container sized and shaped for swallowing by humans.

27. The penetrating member of any previous or subsequent claim, wherein the penetrating member is operatively connected to a pliable substrate.

28. The penetrating member of any previous or subsequent claim, wherein the penetrating member forms a plurality of tissue penetrating drug delivery members in an amount of about 2 to 3000.

29. The penetrating member of any previous or subsequent claim, wherein the microneedle array includes a range of longitudinal sizes ranging between 200-1000 μm.

30. The penetrating member of any previous or subsequent claim, wherein the microneedle array is operatively connected to a pliable substrate which is folded to be contained in a container for oral delivery.

31. The penetrating member of any previous or subsequent claim, wherein the microneedle array is in an amount of 100-150 microneedles per cm2.

32. The penetrating member of any previous or subsequent claim, wherein the active agent and controlled release component is present either: i. as a mixture of active agent and controlled release component; or ii. as a core and coating, wherein the active agent is present as an immediate release active agent inner core and the controlled release component is at least a partial coating on the active agent inner core to form a controlled release coating.

33. The penetrating member of claim 32, wherein the controlled release coating comprises a pH sensitive or sustained release polymers.

34. The penetrating member of claim 32, wherein the coating has a width of between 2 and 25 μm and preferably less than 10 μm.

35. The penetrating member of claim 32, wherein the coating and intestine environment protective component have a combined thickness of less than 50 μm.

36. The penetrating member of claim 32, wherein the immediate release active agent inner core comprises a biodegradable polymer having monomer units for drug delivery.

37. The penetrating member of claim 32, wherein the mixture comprises a controlled release component selected from the list consisting of: polyethylene glycol (PEG), polyvinyl polymers, methylcellulose and ethylcellulose.

38. The penetrating member of claim 37, wherein the ethyl cellulose is a low-viscosity ethyl cellulose.

39. The penetrating member of claim 32, wherein the mixture comprises a low-viscosity ethyl cellulose and a pore-forming agent.

40. The penetrating member of claim 39, wherein the pore forming agent is selected from the group consisting of hydroxypropyl cellulose and hydroxypropyl methylcellulose.

41. The penetrating member of claim 33, wherein controlled release coating comprises any one or more excipients selected from the list consisting of: hydroxypropyl methylcellulose (optionally with ethylcellulose), carboxymethylcellulose, hydroxypropyl cellulose, polyvinyl pyrrolidone (optionally with carboxymethylcellulose), alginates (e.g., sodium alginate), methylcellulose, lipophilic poly-ε-caprolactone and ethyl cellulose.

42. The penetrating member of claim 41, wherein controlled release coating is applied to the immediate release active agent inner core, and said coating is prepared by applying and subsequently drying a formulation selected from the group consisting of: i. carboxymethylcellulose in an amount between 1% and 8%; ii. a hydroxypropyl methylcellulose in an amount of between 2 to 20%; iii. A low-viscosity ethyl cellulose having a viscosity less than about 15 cP and a pore-forming agent selected from the group consisting of hydroxypropyl cellulose and hydroxypropyl methylcellulose; and iv. Polyvinyl pyrrolidone in a concentration between 5% and 30%.

43. The penetrating member claim 41, wherein controlled release coating comprises a low-viscosity ethyl cellulose, a pore-forming agent, and a plasticizer.

44. The penetrating member of claim 39, wherein the pore-forming agent to low-viscosity ethyl cellulose is in a ratio of about 9:1 to about 1:1.

45. The penetrating member of claim 44, wherein the ratio of the pore forming agent to low-viscosity ethyl cellulose is about 7:4 to about 4:5.

46. The penetrating member of claim 40, wherein the viscosity of the hydroxypropyl cellulose or hydroxypropyl methylcellulose is about 3 cP to about 15 cP.

47. The penetrating member of claim 31, wherein the controlled release component is a pH sensitive or sustained release polymer.

48. The penetrating member of claim 33, wherein the sustained release polymer is a non-biodegradable cationic acrylic/methacrylic copolymer.

49. The penetrating member of claim 33, wherein the sustained release polymer is a cationic acrylic/methacrylic copolymer having a high permeability.

50. The penetrating member of claim 33, wherein the sustained release polymer is a cationic acrylic/methacrylic copolymer having a low permeability.

51. The penetrating member of claim 32, wherein the controlled release component further comprises a pore former.

52. The penetrating member of claim 33, wherein the sustained release acrylic/methacrylic copolymer is further coated by an enteric acrylic/methacrylic copolymer.

53. The penetrating member of claim 10, wherein the film-forming polymeric material having a pH threshold at about 6.5 or above is an acrylic/methacrylic copolymer.

54. The penetrating member of claim 53, wherein the film-forming polymeric material is an enteric acrylic/methacrylic copolymer which has a pH threshold of about 7.0 or above.

55. The penetrating member of claim 53, wherein the film-forming polymeric material is an enteric acrylic/methacrylic copolymer which has a pH threshold of about 7.0.

56. The penetrating member of claim 10, wherein the intestine environment protective component is an anionic co-polymer of methacrylic acid and methacrylic acid methyl ester.

57. The penetrating member of claim 56, wherein the acrylic/methacrylic copolymer has a ratio of methacrylic acid to methacrylic acid methyl ester of about 1:2 to 1:1.

58. The penetrating member of claim 57, wherein the acrylic/methacrylic copolymer is an anionic poly(methacrylic acid/methyl methacrylate) copolymer with an acid to ester ratio of 1:2, a molecular weight of approximately 135,000, and a pH threshold is of about 7.

59. The penetrating member of claim 10, wherein the film-forming polymeric material further comprises a second acrylic/methacrylic copolymer having acid: ester ratio about 1:1; MW about 135,000; pH threshold of about 6.0.

60. An oral device for delivery of an active agent across or within a lumen wall of the small intestine of a subject, said oral device comprising:
an intestine release container;
an intestine wall deployment device operably connected to one or more solid tissue penetrating drug delivery members, said deployment device configured to position the penetrating member adjacent to an intestinal lumen wall and apply force to the penetrating member to pierce the lumen wall; and
one or more penetrating members of any previous or subsequent claims, wherein the penetrating members are configured for exposure to the intestinal lumen environment and comprising: a tissue penetrating tip and at least a portion of a therapeutically effective dose of at least one active agent wherein said active agent degrades, is poorly absorbed, and/or is not well tolerated in the GI tract;
wherein the solid penetrating member is configured to be exposed to the intestine lumen environment for a period of time while maintaining size, shape and/or preventing active agent release; and to penetrate and be advanced into the lumen wall by the application of force on the tissue penetrating member, and wherein the solid penetrating member degrades within the lumen wall to release the active agent.

61. An oral device for delivery of an active agent across or within the small intestine lumen wall comprising:
an intestine release container;
a solid controlled-release penetrating member comprising an active agent and having a penetrating tip at a distal end, said tip shaped and formulated to promote penetration of the intestine tissue during exposure to an intestine lumen environment and said penetrating member being operably coupled to an intestine wall deployment assembly; and
an intestine wall deployment assembly disposed in the intestine release container; the assembly configured to:
a. position said penetrating member adjacent to the intestinal lumen wall; and
b. applies a force to said penetrating member so as to penetrate the wall;
wherein when the penetrating member is subject to in vitro dissolution testing employing a USP II apparatus with dissolution medium at 370 C, 30 mM buffer, pH 6.5 with fasted state simulating intestinal fluid (FaSSIF), A. at 10 minutes, i. the penetrating member or more than 80% of the penetrating member array loses less than 20% or 10% of its length, ii. the penetrating member or array of penetrating members release less than 10% of its active agent or iii. the penetrating member or more than 80% of the array of penetrating members maintains a diameter of tip of less than 30 μm or 25 μm; and B. at 120 minutes, the penetrating member releases at least 80% of its active agent.

62. The oral device of any previous or subsequent claim, wherein the penetrating member loses less than 5% of its length at 10 minutes.

63. The oral device of any previous or subsequent claim, wherein the penetrating member loses less than 5% of the active agent at 10 minutes.

64. The oral device of any previous or subsequent claim, wherein the penetrating member has a length of less than 1000 μm measured from a proximal base to a distal tip, wherein the proximal base is operably coupled to an intestine wall deployment assembly.

65. The oral device of claim 64, wherein the penetrating member has a length of about 500 to 850 μm.

66. The oral device of any previous or subsequent claim, wherein the proximal base is operatively connected to a pliable substrate of an intestine wall deployment assembly.

67. The oral device of any previous or subsequent claim, wherein multiple penetrating members are each connected to a common pliable substrate at respective bases of the multiple penetrating members.

68. The oral device of any previous or subsequent claim, wherein the maximum diameter or length at the base of the penetrating member is between 50 and 500 μm.

69. The oral device of any previous or subsequent claim, wherein the penetrating member further comprises a lipophilic coating or a film-forming polymer coating having a pH threshold at about 6.5 or above.

70. The oral device of any previous or subsequent claim, wherein the film-forming polymer is an acrylic/methacrylic copolymer having a pH threshold at about 6.5 or above.

71. The oral device of any previous or subsequent claim, wherein the film-forming polymer coating comprises an enteric acrylic/methacrylic copolymer which has a pH threshold of about 7.0 and a second acrylic/methacrylic copolymer which has a pH threshold of about 6.0.

72. The oral device of any previous or subsequent claim, wherein the penetrating member is one of a plurality of penetrating members.

73. The oral device of any previous or subsequent claim, wherein the plurality of penetrating members forms a microneedle array.

74. The oral device of any previous or subsequent claim, wherein the total amount of active agent in the plurality of penetrating members is between 2 and 10 mg active agent.

75. The oral device of any previous or subsequent claim, wherein the total amount of active agent within the plurality of penetrating members is less than an amount to produce a corresponding effect if the agent was orally delivered in a capsule without the oral device for delivery of active agent across or within a lumen of the small intestine.

76. The oral device of any previous or subsequent claim, wherein the penetrating member has a conical, or pyramid shape.

77. The oral device of any previous or subsequent claim, wherein the solid controlled-release penetrating member comprising the active agent is i. a mixture of active agent and controlled release component; or ii. an immediate release active agent inner core and controlled release coating.

78. The oral device of any previous or subsequent claim, wherein the controlled release coating or controlled release component is a pH sensitive or sustained release polymer.

79. The oral device of any previous or subsequent claim, wherein the controlled release coating is a pH sensitive or sustained release polymer.

80. The solid tissue penetrating drug delivery member of any previous or subsequent claim, wherein the pH sensitive polymer is a cationic acrylic/methacrylic copolymer is non-biodegradable.

81. The oral device of any previous or subsequent claim, wherein the controlled release coating or controlled release mixture is further coated by an enteric acrylic/methacrylic copolymer.

82. The oral device of any previous or subsequent claim, wherein controlled release coating comprises a low-viscosity ethyl cellulose, a pore-forming agent, and a plasticizer.

83. The oral device of any previous or subsequent claim, wherein the assembly applies no force to facing intestinal walls when deployed in the lumen of the intestine and under conditions without intestinal peristalsis.

84. The oral device of any previous or subsequent claim, wherein the tip is at least partially coated, or the tip is formulated as a separate but adjacent and distal extension of the penetrating member, said coat comprising a water insoluble, hydrophobic, or non-degradable polymer.

85. The oral device of any previous or subsequent claim, wherein the microneedle array includes a range of longitudinal sizes ranging between 200-1000 μm.

86. The oral device of any previous or subsequent claim, wherein the microneedle array is in an amount of 100-150 microneedles per cm2.

87. The oral device of any previous or subsequent claim, wherein the penetrating member comprises an outer coating of an intestine environment protective component.

88. The oral device of any previous or subsequent claim, wherein the penetrating member comprises an outer coating of an intestine environment protective component.

89. The oral device of any previous or subsequent claim, wherein the intestine wall deployment assembly comprises:
an expandable member that is associated with one or more solid controlled-release penetrating member, said expandable member having proximal and distal surfaces that face in generally opposite directions and configurable in a compacted configuration, an unconstrained 15 configuration, and an expanded configuration;
a gas generating compartment in fluid communication with the expandable member and having at least a water or humidity permeable window and configured to expand a pliable expandable member from an unconstrained configuration to an expanded configuration; and
one or more solid controlled-release penetrating members, operably coupled to said pliable expandable member and extending from the proximal surface thereof.

90. The oral device any previous or subsequent claim, wherein the array or plurality of microneedles is in an amount of about 800 to 2000 or about 1000 to 1500.

91. The oral device for delivery of active agent across or within a lumen of the small intestine of an active agent across or within the small intestine lumen wall, wherein the intestine wall deployment device comprises i. an expandable member that is associated with one or more tissue penetrating members, said expandable member having proximal and distal surfaces that face in generally opposite directions and configurable in a compacted configuration, an unconstrained configuration, and an expanded configuration; ii. a gas generating compartment in fluid communication with the expandable member and having at least a water or humidity permeable window and configured to expand a pliable expandable member from an unconstrained configuration to an expanded configuration; and iii. one or more solid tissue penetrating member of any precious claim, operably coupled to said pliable expandable member and extending from the proximal surface thereof.

92. An array of any previous or subsequent, wherein the array forms a patch further comprising i. an expandable member that is associated with one or more tissue penetrating members, said expandable member having proximal and distal surfaces that face in generally opposite directions and configurable in a compacted configuration, an unconstrained configuration, and an expanded configuration; ii. a gas generating compartment in fluid communication with the expandable member and having at least a water or humidity permeable window and configured to expand a pliable expandable member from an unconstrained configuration to an expanded configuration; and iii. one or more tissue penetrating members operably coupled to said pliable expandable member and extending from the proximal surface thereof, said penetrating member being sized and configured to penetrate a mucosal barrier of the intestinal wall to release an active agent and comprising: an active agent, an intestine environment protective component for maintaining strength and/or shape and/or resisting active agent release during exposure to the intestinal lumen environment for a period of time and an active agent release component for subsequent release of the active agent across of within the small intestine lumen wall for a period of time to release the active agent.

93. A method of penetrating the mucosal tissue to deliver an active agent comprising:
converting a pliable expandable member from a constrained configuration to an expanded configuration, said expandable member being operably connected to one or more tissue penetrating members in an intestinal protected from comprising a layer or film or pore forming polymer (e.g., a plurality of microneedles) which are exposed to the intestine environment for an exposed period of time; enzymatic, chemical, or mechanical degradation of a layer or film or pore forming polymer to expose an exposed form of the one or more tissue penetrating members; iii. penetrating the mucosal barrier with one or more solid tissue penetrating drug delivery members; and releasing active agent over a release period of time.

94. The method of any previous or subsequent claim 70, wherein the exposed period of time is greater than 5 minutes or less than 15 minutes.

95. The method of any previous or subsequent claim 70, wherein the release period of time is between 15 minutes and 2 hours.

96. A method of delivering an active agent across or within the small intestine lumen wall, the method comprising: deploying a drug delivery device (e.g., patch) adjacent to a small intestine lumen wall said drug delivery device comprising one or more penetrating members (e.g., a plurality of microneedles), a pliable base and an expandable member; exposing the one or more penetrating members (e.g., a plurality of microneedles), pliable base and an expandable member to the intestinal fluid for a period of time; subsequently, penetrating the mucosal tissue with one or more solid tissue penetrating drug delivery members (e.g., a plurality of microneedles) that extend from, or are extendable from, the expandable member at a selected time after the drug delivery device is deployed in the lumen; and releasing an active agent in a controlled manner over a period of between 30 minutes and 4 hours such that the active agent is delivered through the plurality of microneedles into the mucosal tissue or is delivered to the region of the mucosal tissue that is disrupted by the microneedles.

97. A method of delivering an active agent across or within the small intestine lumen wall, the method comprising the steps of:

98. receiving, by a subject, an oral device for delivery of active agent across or within a lumen of the small intestine comprising: (i) an intestine release container and (ii) a drug-delivery device contained within the release component, which includes an active agent formulation shaped as a tissue penetrating formulation and configured to be advanced across or within the small intestine lumen wall;
swallowing the drug delivery device by the subject;
degrading or dissolving the intestine release container in the presence of an intestinal lumen environmental condition;
exposing the solid tissue penetrating drug delivery members to the intestine environment for a period of time while preventing release of the active agent and/or maintaining strength and/or maintaining shape of the tissue penetrating member to advance the at least one tissue penetrating member across or within the small intestine lumen wall tissue; and
release of the active agent into the wall of the small intestine lumen over a period of time

99. A method of delivering an active agent across or within the small intestine lumen wall, the method comprising the steps of:
receiving, by a subject, an oral device for delivery of active agent across or within a lumen of the small intestine comprising: (i) an intestine release container and (ii) a drug-delivery device contained within the release component, which includes an active agent formulation shaped as a tissue penetrating formulation for advancing a tissue penetrating formulation across or within the small intestine lumen wall, the expandable member expanded by gas generating compartment; configured to penetrate a the mucosal barrier of the small intestine lumen wall, an expandable member configurable in a compacted configuration, an unconstrained configuration and an expanded configuration;
swallowing the drug delivery device by the subject;
degrading or dissolving the intestine release container in the presence of an intestinal lumen environmental condition;
exposing the solid tissue penetrating drug delivery members to the intestine environment for a period of time while preventing release of the active agent and/or maintaining strength and/or maintaining shape of the tissue penetrating member to allow for a period of time for the expandable member to reach an expanded configuration to advance the at least one tissue penetrating member across or within the small intestine lumen wall tissue; and
release of the active agent across or within the small intestine lumen wall over a period of time.

100. A method for delivering an active agent across or within the small intestine lumen wall, the method comprising the steps of:
receiving, by a subject, oral device for delivery of active agent across or within a lumen of the small intestine comprising: (i) an intestine release container and (ii) a drug-delivery device contained within the release component, which includes (a) an expandable member for advancing a tissue penetrating formulation across or within the small intestine lumen wall, the expandable member expanded by gas generating compartment; and a tissue penetrating formulation; and swallowing the drug delivery device by the subject;
dissolving the intestine release container (e.g., enteric layer as capsule or coated soft gel capsule) in the small intestine;
exposing a first state of the solid tissue penetrating member having a coating comprising an intestine lumen environment resistant component for a period of time while preventing active agent release and/or maintaining physical strength and/or maintaining shape to allow for a controlled penetration of the second state of the tissue penetrating formulation across or within the small intestine lumen wall said component selected from list comprising a lipophilic or thin film coating, a pore forming component, an interstitial space sensitive biodegradable polymer; and
puncturing the small intestine lumen wall with the second state of the tissue penetrating formulation (e.g., one or a plurality of microneedles), said second state of the tissue penetrating formulation comprising an active agent release component selected from the group comprising: a biodegradable excipient, a pore forming component, an interstitial space sensitive biodegradable polymer); and delivering the active agent across or within the intestine lumen wall.
